(19) United States
(12) Patent Application Publication
(10) Pub. No.: US 2025/0114312 A1
(43) Pub. Date: Apr. 10, 2025

(54) TREATMENT OF COPPER DISORDERS

(71) Applicant: PhilERA New Zealand Ltd., Auckland (NZ)

(72) Inventors: Joseph FORTUNAK, Silver Spring, MD (US); Garth COOPER, Auckland (NZ); Margaret COOPER, Auckland (NZ)

(21) Appl. No.: 18/799,881

(22) Filed: Aug. 9, 2024

Related U.S. Application Data
(63) Continuation of application No. 17/687,525, filed on Mar. 4, 2022, now abandoned.
(60) Provisional application No. 63/157,539, filed on Mar. 5, 2021.

(51) Int. Cl. A61K 31/132; A61K 9/48; A61K 31/352; A61K 31/4525; A61K 45/06; A61P 3/12

(52) U.S. Cl. CPC A61K 31/132; A61K 9/48; A61K 31/352; A61K 31/4525; A61K 45/06; A61P 3/12

(57) ABSTRACT

The present disclosure relates to compositions comprising fixed doses of triethylenetetramine disuccinate, and methods for their use in the prophylaxis and treatment of copper-related diseases, disorders and conditions.

### RELATED APPLICATIONS

This application is a continuation of U.S. application Ser. No. 17/687,525 filed Mar. 4, 2022, which claims the benefit of priority to U.S. Provisional Application No. 63/157,539 filed Mar. 5, 2021, the entire disclosure of which is incorporated herein by reference.

### FIELD

The invention concerns fixed dose triethylenetetramine disuccinate formulations and their use in the treatment, prevention or amelioration of diseases, conditions and disorders treatable with copper chelators.

### INCORPORATION BY REFERENCE

All U.S. patents, U.S. patent application publications, foreign patents, foreign and PCT published applications, articles and other documents, references and publications noted herein, and all those listed as References Cited in any patent or patents that issue herefrom, are hereby incorporated by reference in their entirety. The information incorporated is as much a part of this application as if all the text and other content is repeated in the application and will be treated as part of the text and content of this application as filed.

### BACKGROUND

The following includes information that may be useful in understanding the present inventions. It is not an admission that any of the information is prior art, or relevant, to the presently described or claimed inventions, or that any publication or document that is specifically or implicitly referenced is prior art or a reference that may be used in evaluating patentability of the described or claimed inventions.

Copper is an essential trace element involved in a large number of biological processes in living cells. Analysis of the human proteome has identified 54 copper-binding proteins, of which 12 are copper transporters, approximately half are enzymes and one (Antioxidant 1 Copper Chaperone, ATOX1) is a transcription factor. Copper-binding proteins include cytochrome oxidase, copper-zinc-superoxide dismutase, lysyl oxidase, tyrosinase, and dopamine-beta-monooxygenase, which are involved in pivotal biological processes like mitochondrial respiration, antioxidant defense, extracellular matrix cross-linking, pigmentation and neurotransmitter biosynthesis, respectively. A list of copper-requiring enzymes, with particular emphasis on enzymes involved in genetic disorders of copper homeostasis, may be found in Horn N., et al. Chelating principles in Menkes and Wilson diseases: Choosing the right compounds in the right combinations at the right time. J. Inorg. Biochem. 2019; 190:98-112. The majority of copper in the body is located in organs with high metabolic activity, such as liver, kidneys, heart and brain, with approximately 5% of total copper in the serum, of which up to 95% is bound to ceruloplasmin.

Unbound copper behaves as a potent oxidant, catalyzing the formation of highly reactive hydroxyl radicals leading to DNA, protein and lipid damage. Therefore, cellular copper concentration needs to be finely regulated by complex homeostatic mechanisms of absorption, excretion and bioavailability.

Upon absorption in the gastrointestinal tract, copper reaches the blood, where it is mostly bound to ceruloplasmin. Copper transporter 1 (CTR1, SLC31A1), located on the cell membrane, is the main copper import protein. Within the cell various metallochaperones receive and deliver copper to specific locations. ATPase copper-transporting alpha (ATP7A) and ATPase copper-transporting beta (ATP7B) are key players in copper homeostasis and are required for copper delivery to the secretory pathway and for efflux of excess copper from the cell. Dysregulation of copper homeostasis has been associated with the pathogenesis of several diseases. See Brewer G. J., Copper in medicine. Curr. Opin. Chem. Biol. 2003; 7:207-212; Bandmann O., et al. Wilson's disease and other neurological copper disorders. Lancet Neurol. 2015; 14:103-113.

A chelator is a chemical compound able to selectively bind, due to its structure, a particular atom/ion, generally with the formation of a stable complex ring-like structure. Copper overload toxicity as well as clinically significant copper deficiency are mostly associated with genetic defects of copper transport such as Wilson's disease (copper overload) and Menkes disease (copper deficiency). However, copper is an essential catalytic cofactor in redox biochemistry, and copper dyshomeostasis leading to its unpaired distribution has been linked with several disorders including diabetes, neurological disorders and cancer.

Wilson's disease is an autosomal recessive disease caused by mutations in both copies of the ATP7B gene [18,24] leading to excess copper in the body and characterized by a series of clinical manifestations which include liver failure, tremors and other neurological symptoms. Therefore, to manage increased copper levels, Wilson's disease patients have been treated with different agents that diminish copper, including D-penicillamine (DPA), trientine hydrochloride and tetrathiomolybdate. The goal of copper chelating therapy for Wilson's disease is to remove copper accumulated in tissues (de-coppering phase) and to prevent re-accumulation (maintenance phase). DPA was introduced in 1956. It is a dimethylated cysteine that mobilizes tissue copper stores and promotes excretion of excess copper into urine. DPA (sold as Curprimine® and Depen®) is a first line therapy used to treat Wilson's disease (an inherited condition that causes copper to build up in the body and may result in serious symptoms) and cystinuria (an inherited condition that can lead to kidney stones). However, it has a number of serious side effects, including stomach/abdominal pain, nausea, vomiting, loss of appetite, diarrhea, decreased sense of taste, itching or rash, tinnitus (ringing in the ears), sores in the mouth, poor wound healing, and increased wrinkling of the skin. Additionally, this amelioration of copper balance is not followed by improvements in neurological symptoms. Instead, DPA treatment may be responsible for worsening patients' neurological symptoms, believed to be due to a putative increase in brain copper level. Brewer G. J., et al. Worsening of neurologic syndrome in patients with Wilson's disease with initial penicillamine therapy. Arch. Neurol. 1987; 44:490-493. Furthermore, the use of DPA has been limited by hematologic and renal toxicities (Brewer G. J., Yuzbasiyan-Gurkan V. Wilson disease. Med. (Baltim.) 1992; 71:139-164) and alternative anti-copper agents came into use, such as trientine in 1980. Walshe J. M. Treatment of Wilson's disease with trientine (triethylene tetramine) dihydrochloride. Lancet. 1982; 1:643-647. Triethylenetetramine (TETA), also known as trientine, was specifically introduced as a hydrochloride salt for the treatment of Wilson's patients showing DPA intolerance. Id. Trientine dihydrochloride has an improved safety profile but lower cupreuremic effect compared to DPA.

Alzheimer's disease (AD) is the most common form of dementia, characterized by progressive memory loss, language difficulties, disorientation along with recognizable pathological markers including senile plaques and neurofibrillary tangles. From a molecular point of view, AD is characterized by extracellular deposits of β-amyloid protein accumulated in the brain, ultimately leading to neuronal loss. Condensation of β-amyloid in plaques is linked to high concentrations of Cu(II) and Zn(II) in the neocortical tissue, therefore suggesting a role of metal imbalance in the onset and/or progression of AD. Moreover, β-amyloid binds and reduces Cu(II) to Cu(I), inducing electron transfer to molecular oxygen with the formation of H₂O₂, leading to apoptotic cell death and potentially other negative outcomes associated with oxidative stress. Copper levels in cerebrospinal fluid of AD patients are 2.2 fold higher than in controls, and increased levels of ceruloplasmin in the brain and in cerebrospinal fluid have been observed. Basun H., et al. Metals and trace elements in plasma and cerebrospinal fluid in normal aging and Alzheimer's disease. J. Neural. Transm. Park Dis. Dement. Sect. 1991; 3:231-258. Post-mortem analysis performed on a transgenic mouse model of AD demonstrated that metal chelating agents can attenuate β-amyloid protein excess. Cherny R. A., et al. Treatment with a copper-zinc chelator markedly and rapidly inhibits beta-amyloid accumulation in Alzheimer's disease transgenic mice. Neuron. 2001; 30:665-676; Dedeoglu A., et al. Preliminary studies of a novel bifunctional metal chelator targeting Alzheimer's amyloidogenesis. Exp. Gerontol. 2004; 39:1641-1649. A clinical trial in AD patients using D-penicillamine has been performed. Squitti R., et al. D-penicillamine reduces serum oxidative stress in Alzheimer's disease patients. Eur. J. Clin. Investig. 2002; 32:51-59.

Copper homeostasis alteration also plays a role in Parkinson's disease (PD). PD is among the most common neurodegenerative disorders, affecting approximately 2-3% of the population over 65 years. The principal hallmark of PD is represented by the typical dopamine-producing neuronal loss in the substantia nigra, accompanied by α-synuclein aggregates usually termed as Lewy bodies, leading to the characteristic symptoms of bradykinesia, muscular rigidity, tremors and other non-motor symptoms. Copper binding to the α-synuclein protein is an important event in the development of PD, triggering protein fibrillation and increased oxidative stress. Moreover, the binding of copper to ceruloplasmin is reduced in PD patients, leading to an increase in the levels of free copper, associated with oxidative stress and neurodegeneration. Ajsuvakova O. P., et al. Assessment of copper, iron, zinc and manganese status and speciation in patients with Parkinson's disease: A pilot study. J. Trace Elem. Med. Biol. 2019:126423. Therefore, copper homeostasis alteration plays a role in PD. Bjorklund G., et al. Metals and Parkinson's disease: Mechanisms and biochemical processes. Curr. Med. Chem. 2018; 25:2198-2214. Copper attenuation therapy in PD was recently reviewed by Tosato, M and Di Marco, V, in Metal chelation therapy and Parkinson's disease: A critical review on the thermodynamics of complex formation between relevant metal ions and promising or established drugs. Biomolecules. 2019; 9:269.

Idiopathic pulmonary fibrosis (IPF) is a form of chronic lung disease, usually affecting people between the ages of 50 and 80 years, in which fibrosis progressively build up in the lungs, leading to impairment of lung functions. The wide heterogeneity of clinical manifestations and symptoms leads to a high variability in therapy course and response. The exact mechanism of IPF pathogenesis has not been clarified yet, however different biological and molecular factors may be involved including lysyl oxidases, a group of copper-dependent enzymes involved in covalent cross-linking of type I collagen. In particular, LOXL2 may represent a potential therapeutic target, being pro-fibrotic and highly expressed in IPF lung biopsies. See, e.g., Chen L., et al. LOX/LOXL in pulmonary fibrosis: Potential therapeutic targets. J. Drug Target. 2019; 27:790-796. A study, performed in 2003, demonstrated that administration of the copper chelator tetrathiomolybdate induced a reduction in serum ceruloplasmin leading to a corresponding reduction of lung fibrosis in a mouse model of bleomycin-induced IPF, paving the way for a clinical trial on IPF patients unresponsive to other therapies. Brewer G. J., et al. Tetrathiomolybdate therapy protects against bleomycin-induced pulmonary fibrosis in mice. J. Lab. Clin. Med. 2003; 141:210-216. It has been proved that copper chelation with tetrathiomolybdate exerts its beneficial effect on IPF by reducing collagen-I expression and accumulation, acting on the expression of the copper-dependent lysyl oxidases. Ovet H., Oztay F. The copper chelator tetrathiomolybdate regressed bleomycin-induced pulmonary fibrosis in mice, by reducing lysyl oxidase expressions. Biol. Trace Elem. Res. 2014; 162:189-199.

Diabetes mellitus (DM) is a group of heterogeneous metabolic diseases mainly characterized by a hyperglycemic condition, with a defect in insulin secretion or action. There are three main types of diabetes: type I, type II and gestational diabetes. DM patients have higher levels of copper in plasma or serum compared to healthy individuals. Qiu Q., et al. Copper in diabetes mellitus: A meta-analysis and systematic review of plasma and serum studies. Biol. Trace Elem. Res. 2017; 177:53-63; Li P., et al. Association between plasma concentration of copper and gestational diabetes mellitus. Clin. Nutr. 2019; 38:2922-2927. The development and progression of DM have been associated with an increase in oxidative stress and with imbalance of several metals, including copper. Zheng Y., et al. The role of zinc, copper and iron in the pathogenesis of diabetes and diabetic complications: Therapeutic effects by chelators. Hemoglobin. 2008; 32:135-145. Transition between Cu(I) and Cu(II) leads to the production of reactive oxygen species (ROS) and to consequent peroxidation of lipids, DNA damage leading to cell death. Copper homeostasis maintenance using copper chelators may represent a strategy for diabetes treatment. See Lowe J., et al. Dissecting copper homeostasis in diabetes mellitus. Iubmb Life. 2017; 69:255-262.

Importantly, a series of pre-clinical and clinical studies with the hydrochloride salt of triethylenetetramine demonstrated the potential of the copper chelator trientine in reducing some of the clinical and pathological consequences of diabetes, such as heart failure. Cooper G. J., Phillips A. R., Choong S. Y., Leonard B. L., Crossman D. J., Brunton D. H., Saafi L., Dissanayake A. M., Cowan B. R., Young A. A., et al. Regeneration of the heart in diabetes by selective copper chelation. Diabetes. 2004; 53:2501-2508. Additionally, treatment with the copper chelator tetrathiomolybdate was described to promote a significant reduction of insulin resistance in a mouse model of type 2 diabetes. Tanaka A., et al. Role of copper ion in the pathogenesis of type 2 diabetes. Endocr. J. 2009; 56:699-706.

Copper is also related to cancer, where increased copper content has been determined in serum (Coates R. J., et al. Cancer risk in relation to serum copper levels. Cancer Res. 1989; 49:4353-4356) and tissue samples (Margalioth E. J., et al. Copper and zinc levels in normal and malignant tissues. Cancer. 1983; 52:868-872) from patients with different types of cancer, including laryngeal squamous cell carcinoma (de Jorge F. B., et al. Biochemical studies on copper, copper oxidase, magnesium, sulfur, calcium and phosphorus in cancer of the larynx. Acta Otolaryngol. 1966; 61:454-458), non-Hodgkin's lymphoma (Shah-Reddy I., et al. Serum copper levels in non-Hodgkin's lymphoma. Cancer. 1980; 45:2156-2159), multiple myeloma (Khadem-Ansari M. H., et al. Copper and zinc in stage I multiple myeloma: Relation with ceruloplasmin, lipid peroxidation, and superoxide dismutase activity. Horm. Mol. Biol. Clin. Investig. 2018:37), chronic lymphocytic leukemia (Kaiafa G. D., et al. Copper levels in patients with hematological malignancies. Eur. J. Intern. Med. 2012; 23:738-741), hepatocellular carcinoma (Fang A. P., et al. Serum copper and zinc levels at diagnosis and hepatocellular carcinoma survival in the Guangdong Liver Cancer Cohort. Int. J. Cancer. 2019; 144:2823-2832), gynecological carcinoma (Zowczak M., et al. Analysis of serum copper and zinc concentrations in cancer patients. Biol. Trace Elem. Res. 2001; 82:1-8), colorectal (Gupta S. K., et al. Serum and tissue trace elements in colorectal cancer. J. Surg. Oncol. 1993; 52:172-175), lung (Zhang X., Yang Q. Association between serum copper levels and lung cancer risk: A meta-analysis. J. Int. Med. Res. 2018; 46:4863-4873), primary brain (Turecký L., et al. Serum ceruloplasmin and copper levels in patients with primary brain tumors. Klin. Wochenschr. 1984; 62:187-189), and breast (Dabek J. T., et al. Evidence for increased non-ceruloplasmin copper in early-stage human breast cancer serum. Nutr. Cancer. 1992; 17:195-201) cancers. Serum copper levels return to normal upon successful tumor surgical removal or on remission. In addition, gene expression analysis revealed multiple alterations in a variety of copper-binding or copper-sensitive proteins in colorectal (Barresi V., et al. Transcriptome analysis of copper homeostasis genes reveals coordinated upregulation of SLC31A1, SCO1, and COX11 in colorectal cancer. FEBS Open Bio. 2016; 6:794-806) and breast cancers (Nagaraja G. M., et al. Gene expression signatures and biomarkers of noninvasive and invasive breast cancer cells: Comprehensive profiles by representational difference analysis, microarrays and proteomics. Oncogene. 2006; 25:2328-2338), suggesting that deregulation of copper homeostasis might contribute to cancer pathogenesis, development and metastasis.

Collectively these indications provide support for copper chelation as strategies for cancer therapy. See Goodman V. L., et al. Copper deficiency as an anti-cancer strategy. Endocr. Relat. Cancer. 2004; 11:255-263; Lopez J., et al. Copper depletion as a therapeutic strategy in cancer. Met. Ions Life Sci. 2019:19; Garber K. Cancer's copper connections. Science. 2015; 349:129. Neoangiogenesis is essential to support cancer cells growth and tumor metastasis. The mechanism of cancer inhibition by copper chelating agents is commonly attributed to their inhibitory effect on tumor angiogenesis. Goodman V. L., et al., supra. Indeed, copper chelating agents used to treat Wilson's disease such as trientine, revealed chemotherapeutic properties in experimental preclinical cancer models, leading to several clinical trials. These trials have proved that copper chelation therapy is generally well tolerated, for the reason that copper chelation agents act selectively on cancer cells, which have increased copper content, exerting little toxicity to normal cells. Denoyer D., et al. Targeting copper in cancer therapy: ‘Copper That Cancer’ Metallomics. 2015; 7:1459-1476; Gupte A., Mumper R. J. Elevated copper and oxidative stress in cancer cells as a target for cancer treatment. Cancer Treat. Rev. 2009; 35:32-46.

It is also noted that increased efficacy of radiotherapy against primary tumors with reduced side effects can be achieved when combined with antiangiogenic agents, and an additive effect of radiotherapy and copper chelation therapy was observed in a Lewis lung high metastatic carcinoma mouse tumor model. Khan M. K., et al. Combination tetrathiomolybdate and radiation therapy in a mouse model of head and neck squamous cell carcinoma. Arch. Otolaryngol. Head Neck Surg. 2006; 132:333-338.

Copper chelation and immunotherapy combination strategies have also been proposed and evaluated for use with several immunotherapy strategies, including monoclonal antibodies, immune cell activators, immune checkpoint inhibitors and oncolytic viral vectors. For example, the strategy of nanoparticle-based copper chelation and immune stimulation has been shown to effectively inhibit breast tumor growth and metastasis in experimental models both in vitro and in vivo. Zhou P., et al. Multifunctional nanoparticles based on a polymeric copper chelator for combination treatment of metastatic breast cancer. Biomaterials. 2019; 195:86-99. With regard to immune checkpoint inhibitors, a positive correlation between the copper transport protein CTR1 and programmed cell death protein 1 (PD-1) expression has been observed in neuroblastoma and glioblastoma tumor cells. Copper chelation reduces PD-L1 expression, promoting a significant increase in tumor-infiltrating lymphocytes in a syngeneic mouse model of neuroblastoma. Voli F., et al. Copper homeostasis: A new player in anti-tumor immune response. Cancer Res. 2019:79. Therefore, copper chelation therapy may also promote the efficacy of programmed cell death protein 1 (PD-1)/programmed cell death ligand 1 (PD-L1) based immunotherapy. Oncolytic vectors selectively replicate and promote lysis of cancer cells triggering the patient's immune system against tumor antigens. Changes in the tumor microenvironment in response to induced oncolysis may limit the efficacy of oncolytic virotherapy. Therefore, is has been hypothesized that combination of copper chelation therapy, which affects both tumor microenvironment and angiogenesis, may promote the efficacy of oncolytic virotherapy. In addition, serum copper levels have a detrimental effect on herpes virus infection. Based on these premises, it has been described that concomitant copper chelation therapy increases antitumor effect of herpes simplex virus-derived oncolytic viruses. Yoo J. Y., et al. Copper chelation enhances antitumor efficacy and systemic delivery of oncolytic HSV. Clin. Cancer Res. 2012; 18:4931-4941; Yoo J. Y., et al. ATN-224 [Bis-Choline Tetrathiomolybdate] enhances antitumor efficacy of oncolytic herpes virus against both local and metastatic head and neck squamous cell carcinoma. Mol. Oncolytics. 2015; 2:15008.

Autophagy has complex role in cancer development, progression and response to therapy. Autophagy inhibition is emerging as an effective approach for tumor therapy, particularly in cancers with increased levels of basal autophagy. Amaravadi R. K., et al. Targeting autophagy in cancer: Recent advances and future directions. Cancer Discov. 2019; 9:1167-1181. Different lines of evidence suggest that increased copper content activates a series of autophagy-related genes. Polishchuk E. V., et al Activation of autophagy, observed in liver tissues from patients with Wilson disease and from ATP7B-deficient animals, protects hepatocytes from copper-induced apoptosis. Gastroenterology. 2019; 156:1173-1189.e1175. Accordingly, copper chelation has been shown to inhibit the Unc-51-like autophagy activating kinase 1 and 2 (Ulk1/2) in lung adenocarcinoma cells. Tsang T., et al. Copper is an essential regulator of the autophagic kinases ULK1/2 to drive lung adenocarcinoma. bioRxiv. 2019:816587. Recently, the combination of copper chelation and autophagy inhibition by chloroquine has been evaluated to promote pancreatic cancer cells death. Yu Z., et al. Blockage of SLC31A1-dependent copper absorption increases pancreatic cancer cell autophagy to resist cell death. Cell Prolif. 2019; 52:e12568.

In addition to the use of triethylenetetramine dihydrochloride as therapy for treating individuals with Wilson's disease, it has also reportedly been used to treat individuals with primary biliary cirrhosis. See, e.g., Epstein, O. et al., Gastroenterology 78(6):1442-45 (1980). In addition, trientine has been tested for inhibition of the spontaneous development of hepatitis and hepatic tumors in rats. See, e.g., Sone, H., et al., Hepatology 23:764-70 (1996). Issued United States patents describe the use of copper binding compounds in the treatment of various disorders, including treatment of diabetes mellitus and complications thereof, including, for example, diabetic cardiomyopathy. See U.S. Pat. Nos. 6,897,243, 6,610,693 and 6,348,465. Certain copper chelators have also been described for use in treating certain disorders, including cardiovascular, glucose and vascular disorders. Prior teachings relating to copper chelators are described in, for example, U.S. Pat. No. 10,543,178 (use of a succinic acid addition salt of triethylenetetramine to treat diabetic neuropathy), U.S. Pat. No. 9,993,443 (use of a succinic acid addition salt of triethylenetetramine to treat tissue damage associated with specific cardiac, glucose related and vascular disorders), U.S. Pat. No. 8,987,244 (use of various chelators, including trientine, 2,2,2 tetramine tetrahydrochloride and 2,3,2 tetramine tetrahydrochloride to lower copper(II) values in patients with tissue damage in myocardial tissue, kidney tissue, eye tissue, nerve tissue, and vascular tissue), U.S. Pat. No. 8,563,538 (use of 2,3,2 tetramine compositions in methods of treating heart failure in a non-diabetic human subject, including 2,3,2 tetramine hydrochloride salts, e.g., 2,3,2 tetramine tetrahydrochloride), U.S. Pat. No. 8,034,799 (methods of treating heart failure in a non-diabetic human subject with an agent capable of reducing copper levels, for example, copper(II), including copper chelators such as trientine, as well as 2,3,2 tetramine, DPA, N-acetylpenicillamine, trithimolybdate, and tetrathimolybdate), and U.S. Pat. No. 7,928,094 (use of triethylenetetramine dihydrochloride to treat one or more conditions associated with long-term complications of diabetes).

In sum, a growing number of in vitro and in vivo studies suggest that copper-involving mechanisms represent a potential therapeutic target for a number of different pathologies. A state of systemic or tissue-specific copper increase can occur through multiple mechanisms in addition to the genetic defects of copper metabolism observed in Wilson's disease. Dysregulation of copper homeostasis has been observed in a wide spectrum of neurological, fibrotic pulmonary and vascular diseases as well as in different types of cancers.

Copper imbalance in Wilson's disease has been well investigated, leading to the introduction of copper chelation therapy as a primary therapeutic tool which has significantly reduced morbidity, making Wilson's disease a treatable disorder. As noted, treatment with first-in-line DPA therapy has a number of side effects, including stomach/abdominal pain, nausea, vomiting, loss of appetite, diarrhea, decreased sense of taste, itching or rash, tinnitus (ringing in the ears), sores in the mouth, poor wound healing, and increased wrinkling of the skin. Trientine hydrochloride (sold as the dihydrochloride salt, under the trade names Syprine® and Clovique®) chelates copper and is used to treat Wilson's disease in people who cannot take penicillamine. Syprine® was approved by the FDA in 1985 as a second line treatment for Wilson's Disease. Common side effects of trientine dihydrochloride include skin rash, muscle spasm or contractions, heartburn, stomach pain, loss of appetite and skin flaking, cracking, or thickening.

Efforts have been focused on identifying and evaluating new chelating compounds and formulations to reduce toxic side effects, enhance ability to pass through the blood-brain barrier and improve patient's compliance, and there exists a need to develop improved methods of treating subjects with copper disorders. The present disclosure satisfies these needs and provides methods and compositions to reduce copper and chelator side effects, improve drug stability and avoid the requirement for cold storage and cold chain distribution.

### BRIEF SUMMARY

The inventions described and claimed herein have many attributes and embodiments including, but not limited to, those set forth or described or referenced in this Brief Summary. It is not intended to be all-inclusive and the inventions described and claimed herein are not limited to or by the features or embodiments identified in this introduction, which is included for purposes of illustration only and not restriction.

The invention relates to improved, fixed dose amounts of triethylenetetramine disuccinate, formulations thereof, and their use for the treatment, prevention or amelioration of diseases, conditions and disorders treatable with copper chelators.

In one aspect, the invention comprises an article of manufacture comprising a single dose capsule or tablet containing a single fixed dose of triethylenetetramine disuccinate, wherein the fixed dose is selected from the group consisting of about 350 mg, 400 mg, about 500 mg, about 600 mg and about 700 mg of triethylenetetramine disuccinate.

In another aspect, the article of manufacture further comprises a package insert instructing the user to administer the fixed dose to a patient with a disease, condition or disorder treatable with a copper chelator. In a further aspect, the disease treatable with a copper chelator is characterized by excess copper.

In another aspect, the disease, condition or disorder to be treated as describe herein is selected from the group consisting of Wilson's Disease, heart failure, diabetic cardiomyopathy, left ventricular hypertrophy, diabetes mellitus, Alzheimer's Disease, Parkinson's Disease, idiopathic pulmonary fibrosis, and cancer. In other embodiments, the disease, condition or disorder to be treated as describe herein is selected from the group consisting of fronto-temporal dementia (FTD), multiple sclerosis, and amyotrophic lateral sclerosis (Lou Gehrig's disease/motor neuron disease, or ALS).

In another aspect, the disease, condition or disorder is copper toxicity.

In another aspect, the cancer is selected from the group consisting of laryngeal squamous cell carcinoma, non-Hodgkin's lymphoma, multiple myeloma, chronic lymphocytic leukemia, hepatocellular carcinoma, gynecological carcinoma, colorectal carcinoma, lung cancer, primary brain cancer, and breast cancers.

In another aspect, the fixed dose of triethylenetetramine disuccinate is used to inhibit tumor angiogenesis.

In still another aspect, the fixed dose of triethylenetetramine disuccinate is used with radiotherapy against tumors and cancers, including Lewis lung high metastatic carcinoma.

In yet another aspect, the fixed dose of triethylenetetramine disuccinate is used with immune stimulation, including to inhibit breast tumor growth and metastasis.

In another aspect, the fixed dose of triethylenetetramine disuccinate is used to reduce programmed cell death protein 1 (PD-1) expression, which has been observed, for example, in neuroblastoma and glioblastoma tumor cells.

In another aspect, the fixed dose of triethylenetetramine disuccinate is used in combination with oncolytic virotherapy, including oncolytic HSV therapy. In still another aspect, the fixed dose of triethylenetetramine disuccinate is used in combination with oncolytic virotherapy, for example, against local and metastatic head and neck squamous cell carcinomas.

In another aspect, the fixed dose of triethylenetetramine disuccinate is used in combination with an inhibitor of N-acetylaminotransferase. In another aspect, the fixed dose of triethylenetetramine disuccinate is used in combination with an inhibitor of spermidine-spermine-N (1)-acetyltransferase (SSAT1 and/or SSAT2). In one preferred embodiment, the fixed dose of triethylenetetramine disuccinate is used in combination with an inhibitor of spermidine-spermine-N (1)-acetyltransferase-2 (SSAT2).

In still another aspect, the fixed dose of triethylenetetramine disuccinate is used to prevent, treat or manage autophagy.

In another aspect, the article of manufacture comprises a number of capsules equal to a daily dose of triethylenetetramine disuccinate, wherein the daily dose is selected from the group consisting of from about 2400 mg per day to about 3000 mg per day of triethylenetetramine disuccinate. In another aspect, doses and dosing are between about 2.336 and 2.337 mg of triethylenetetramine disuccinate for every milligram of triethylenetetramine dihydrochloride or triethylenetetramine tetrahydrochloride.

In another aspect, the triethylenetetramine disuccinate in the article of manufacture of has a purity of at least about 95%. In a further aspect, the purity is at least about 99%.

In another aspect, the triethylenetetramine disuccinate in the article of manufacture is a crystalline form of triethylenetetramine disuccinate.

In another aspect, the triethylenetetramine disuccinate in the article of manufacture is a triethylenetetramine disuccinate anhydrate.

In another aspect, the triethylenetetramine disuccinate in the article of manufacture is non-hygroscopic and possesses good stability under conditions of normal, room temperature storage. Importantly, the crystalline anhydrous form of the triethylenetetramine disuccinate article of manufacture described herein has a shelf-life of at least about 12 months (and up to five years) at room temperature, without significant degradation of the triethylenetetramine disuccinate API and remains within impurity specifications for the triethylenetetramine disuccinate drug substance. In one embodiment, the term “without significant degradation” means that the purity of the triethylenetetramine disuccinate is at least about 98.5% with no degradation product above about 0.5% and no new, unidentified impurities above about 0.1% for at least about 12 months.

In another aspect, the article of manufacture with a fixed dose of triethylenetetramine disuccinate is in the form of a capsule. In another aspect, the article of manufacture with a fixed dose of triethylenetetramine disuccinate is in the form of a tablet. In a further aspect, the capsule or tablet of triethylenetetramine disuccinate is formulated in a manner so as to provide delayed or sustained release, thereby resulting in a modified pharmacokinetic profile from a related immediate-release form.

In a still further aspect, the invention also comprises a method of managing or treating a subject with a disease treatable with a copper chelator, the method comprising administering triethylenetetramine disuccinate to said subject in an amount ranging from about 2400 mg per day to about 3000 mg per day of triethylenetetramine disuccinate. In one aspect of the method, the disease treatable with a copper chelator is characterized by excess copper. In another aspect, the triethylenetetramine disuccinate used in the methods is at least about 95% pure, at least about 99% pure, or 100% pure. In another aspect, the triethylenetetramine disuccinate used in the method is a crystalline form of triethylenetetramine disuccinate. In yet another aspect of this method, the triethylenetetramine disuccinate is a triethylenetetramine disuccinate anhydrate. In still another aspect of the method the triethylenetetramine disuccinate is in the form of a fixed dose tablet or capsule. In one preferred embodiment, the fixed dose of triethylenetetramine disuccinate is about 400 mg, about 500 mg, about 600 mg or about 700 mg. In another preferred embodiment of the method the subject is a human.

In another embodiment, three fixed dose tablets or capsules of the 400 mg fixed dose of triethylenetetramine disuccinate is given twice per day (2400 mg per day).

In another embodiment, three fixed dose tablets or capsules of the 500 mg fixed dose of triethylenetetramine disuccinate is given twice per day (3000 mg per day).

In another embodiment, the triethylenetetramine disuccinate fixed dose tablets or capsules are 350 mg.

In another embodiment, the total amount given per day is 2800 mg as four 350 mg tablets or capsules BID.

In another aspect, the fixed dose of triethylenetetramine disuccinate is used to lower or normalize copper(II) content in a subject. In one embodiment, the fixed dose of triethylenetetramine disuccinate reduces total copper in the subject. In another embodiment, the fixed dose of triethylenetetramine disuccinate is used to treat a subject for a disease, disorder or condition who would benefit from a copper(II) chelator.

In one preferred embodiment of methods of the invention, fixed dose of triethylenetetramine disuccinate is delivered orally.

In another embodiment, a fixed dose of triethylenetetramine disuccinate maintains total copper in the subject within the normal human serum or plasma range of about 0.8-1.2 milligrams/L, or about 10-25 micromoles/L. In another embodiment, the fixed dose of triethylenetetramine disuccinate maintains total copper in the subject within at least about 70% of the normal range of about 0.8-1.2 milligrams/L or about 10-25 micromoles/L, e.g., at least about 75%. In another embodiment, fixed dose of triethylenetetramine disuccinate maintains total copper in the subject within about 75% to about 85%, or about 85% to about 95% the normal range of copper in human plasma or serum. In one aspect of the methods of the invention, the copper status of a subject given a fixed dose of triethylenetetramine disuccinate is determined by evaluating copper in the urine of the subject.

In one aspect of the invention, the method employs a pharmaceutical composition comprising a fixed dose of substantially pure triethylenetetramine disuccinate. In another aspect the method employs a pharmaceutical composition comprising substantially pure triethylenetetramine disuccinate and a pharmaceutically acceptable excipient.

In one aspect of the invention, the method employs a fixed dose of a crystalline form of triethylenetetramine disuccinate.

In another aspect of the invention, the method employs a fixed dose of triethylenetetramine disuccinate anhydrate.

In certain embodiments, the fixed dose of triethylenetetramine succinate is a triethylenetetramine disuccinate polymorph.

A preferred pharmaceutical composition for use in the methods of the invention comprises or consists essentially of or consists of a fixed dose of substantially pure triethylenetetramine disuccinate. Another preferred composition is a fixed dose of substantially pure triethylenetetramine disuccinate anhydrate. Another preferred composition is a composition that comprises or consists essentially of or consists of a fixed dose of a substantially pure triethylenetetramine disuccinate crystal having alternating layers of triethylenetetramine molecules and succinate molecules.

In another aspect of the invention, the method maintains copper levels with about 70% to about 100% of normal in the subject, thereby eliciting by a lowering of copper values in a mammalian patient and/or reducing the level of copper.

The total dosage of triethylenetetramine disuccinate may be given in single or divided dosage units (e.g., BID, TID), and preferably maintain normal urine and/or plasma copper levels in a subject, or levels that do not fall below about 70% to 75% of normal. Fixed doses of triethylenetetramine disuccinate are typically administered BID.

In some embodiments, the method comprises or consists essentially of or consists of administering a tablet or capsule comprising a fixed dose of triethylenetetramine disuccinate to a subject. Preferably, the fixed dose of triethylenetetramine disuccinate is administered orally in the form of a capsule.

In any of the procedures described and/or claimed herein, the fixed triethylenetetramine disuccinate dosage regimen given to a subject will not reduce physiological levels of copper down to a depletion state or to an otherwise dangerously low level in the subject.

The invention also includes an article of manufacture, e.g., a kit of parts, comprising or consisting essentially of one or more of the fixed doses of triethylenetetramine disuccinate described herein, for example, oral fixed doses of triethylenetetramine disuccinate, and a printed set of instructions (e.g., a package insert) describing their use in therapy, for example in the treatment of heart failure, diabetic cardiomyopathy, left ventricular hypertrophy, Wilson's disease, cancer, etc. In one embodiment, the kit does not include a physical set of instructions, but refers to or describes their availability online, in the cloud, in a flash drive, or another storage mechanism. In one embodiment, the instructions recite that the triethylenetetramine disuccinate is to be administered to patients with Wilson's disease previously receiving triethylenetetramine dihydrochloride or DPA.

Both the foregoing summary and the following detailed description are exemplary and explanatory. They are intended to provide further details of the invention but are not to be construed as limiting. Other objects, advantages, and novel features will be apparent to those skilled in the art from the following detailed description of the invention.

### BRIEF DESCRIPTION OF DRAWINGS

FIG. 1 is a schematic diagram of the final PK/PD model used to describe TETA, MAT, and DAT plasma concentrations and urinary copper excretion versus time in Example 3. Symbols are defined in the List of Abbreviations and in Table 8.

### DETAILED DESCRIPTION

Various methods of formulation are practiced to achieve desired outcomes that maximize the pharmacokinetic profile of absorption, distribution, metabolism, and elimination for drugs. The molecule triethylenetetramine is basic because of the four nitrogen atoms each possessing a lone electron pair. It is a colorless, oily liquid but, like many amines, assumes a yellowish color due to impurities resulting from air-oxidation. It is soluble in polar solvents.

The dihydrochloride salt of triethylenetetramine is classified as a BCS Class III drug (high solubility, low permeability). It is sold under brand names Syprine® (250 mg), Clovique® (250 mg) and Cufence (300 mg). Triethylenetetramine is also available in a tetrahydrochloride salt form marketed as Cuprior® (150 mg). Little to nothing has previously been known about the relative bioavailability of these different salt forms, aside from the high variability in oral bioavailability of triethylenetetramine dihydrochloride salt form (see, e.g., Lu, Jun, Triethylenetetramine Pharmacology and its Clinical Applications, Molecular Cancer Therapeutics Volume 9, Issue 9, pp. 2458-2467 (September 2007), and nothing was known about the bioavailability of the triethylenetetramine disuccinate salt, which we have determined is a BCS Class I drug (high solubility, high permeability) and not a BCS Class III drug.

The triethylenetetramine dihydrochloride salts sold as Syprine® and Clovique® are recommended to be taken on an empty stomach in doses of 750-1250 mg/day given in divided doses to adults between two- and four-times daily with a maximum dose of 2000 mg/day. This product is provided in capsule form, in doses of 250 mg/capsule (contains the equivalent of 200 mg of triethylenetetramine). The Cufence® triethylenetetramine dihydrochloride salt product is sold as 200 mg capsules and the recommended dose is 800-1600 mg (4-8 capsules) daily in 2 to 4 divided doses.

Syprine® was approved for medical use in the United States in November 1985 for the treatment of Wilson's disease. Cloviquie® was approved in the United States in October 2019 for treating Wilson's disease. Cufence® was approved for medical use in Wilson's Disease patients by the European Union in July 2019. Cloviquie® is the first FDA-approved trientine product in a portable blister pack that offers room temperature stability for up to 30 days, potentially providing patients more convenience. Cuprior™ is the tetrahydrochloride salt of triethylenetetramine. This product is provided in 150 mg strength tablets, with each tablet containing the equivalent of 75 mg of triethylenetetramine. It was approved by the European Union for the treatment of Wilson's disease in September 2017. All of these products are formulated for immediate release.

One object of this invention is to provide doses and dosage forms of triethylenetetramine disuccinate that are equivalent in human exposure/bioavailability with previously approved forms of triethylenetetramine for the treatment of, for instance, Wilsons Disease.

Triethylenetetramine disuccinate is an alternative, superior salt form of trientine. It is more stable with better distribution and good activity. It is markedly preferably over the triethylenetetramine dihydrochloride compounds currently used for treating Wilson's Disease in terms of its resistance to light, temperature and moisture. It does not require cold chain storage or special packaging as does the dihydrochloride salt. However, appropriate dosing is unknown.

Evaluation of dosing is complex, based on a number factors, including drug molecular weight, drug stability and half-life, drug solubility, drug permeability across mucosal barriers, bioavailability (availability of drug to the general circulation or site of pharmacological action), tissue distribution and clearance, tissue: blood ratios of drug, binding to plasma proteins, activity by dose, breakdown into active metabolites, average plasma concentration, and excretion routes, as well as the need for bolus or loading doses and/or sustained or basal drug levels. Aside from molecular weight, most of these variables are unknown for the copper chelator triethylenetetramine disuccinate.

Optimal fixed dosing of triethylenetetramine disuccinate for the treatment of diseases has been discovered and is provided herein following the studies described in the below Examples, including the Example 1 intestinal absorption study and the Example 2 in vivo distribution study as well as the human clinical study results described, interpreted and evaluated in Examples 3 and 4, which revealed unexpected findings including those on the copper chelation activity and breakdown of triethylenetetramine disuccinate into major metabolites, average plasma concentration, full body tissue distribution, tissue: blood ratios following oral administration, and triethylenetetramine disuccinate bioavailability, amongst other things.

Example 1 describes an in vitro intestinal absorption study showing that triethylenetetramine disuccinate will have good absorption in humans (estimated at approximately 70%).

Example 2 is a quantitative in vivo study on the tissue distribution of the labelled copper-depriving compound triethylenetetramine disuccinate following oral administration to male albino and male pigmented rats. Significant tissue penetration was found throughout 42 different body tissues, including the brain, heart, lung and liver in both species. In the male pigmented rat, maximum tissue concentrations of radioactivity were evenly distributed between the 1 h and 8 h time points. Highest levels of radioactivity were seen in the various tissues that included the lung at 1 hr post-dose, with penetration to the lung continuing for a full 8 hours. At 24 h post-dose elimination was on-going in the male pigmented rat with approximately half of the measured tissues having levels of radioactivity below the limit of quantification. At 72 h post-dose, elimination of radioactivity in the male pigmented rat was almost complete with approximately 65% of tissues below the limit of quantification.

Example 3 describes human population pharmacokinetic and pharmacodynamic modeling of triethylenetetramine, its two major metabolites, and copper excretion after oral 2-way crossover administration of triethylenetetramine disuccinate and triethylenetetramine dihydrochloride in a clinical study to healthy adult volunteers, revealing, amongst other things, the bioavailability of triethylenetetramine disuccinate in humans. The population PK analysis encompassed samples from this study where each subject received triethylenetetramine disuccinate and triethylenetetramine dihydrochloride (Syprine®) in a double-blind, dose escalation, 2-way crossover design.

Example 4 describes further analyses of data obtained in the Example 2 study comparing triethylenetetramine disuccinate and triethylenetetramine dihydrochloride (Syprine®). The Example 2 study resulted in the discovery that administration of triethylenetetramine as the disuccinate salt results in lower exposure indices (Cmax and AUC) of triethylenetetramine and its metabolites. The modeling in Example 3 compared the absorption kinetics and provided a more global assessment of relative bioavailability of the two salt forms in the context of the Example 3 study design. The Example 4 analysis applied a model-based population analysis to the data in order to obtain an integrated assessment of the pharmacokinetics of triethylenetetramine and its two major metabolites (monoacetylated (MAT) and diacetylated (DAT forms) and to further assess the pharmacodynamics of urinary excretion of copper, to consider potential covariates with the PK/PD parameters such as sex, age and dose, and in comparing the PK/PD of Syprine® and triethylenetetramine disuccinate from the Example 2 bioequivalency study, particularly in regard to bioavailability.

Triethylenetetramine dihydrochloride is a copper chelator that was approved by the FDA for the second line treatment of Wilson's Disease. It is available in Europe in 300 mg capsules and in general two capsules are administered BID (1200 mg per day total) to treat Wilson's Disease. Triethylenetetramine dihydrochloride (Syprine®) is available in the United States in 250 mg capsules and in general two capsules are administered BID (1000 mg per day total) to treat Wilson's Disease. Systemic evaluation of Syprine® dose and/or interval between doses has not been done. However, on limited clinical experience, the recommended initial dose of Syprine® in the United States is 500-750 mg/day for pediatric patients and 750-1250 mg/day (up to 2000 mg/day) for adults given in divided doses two, three or four times daily.

Triethylenetetramine disuccinate is an alternative, superior salt form of triethylenetetramine, but its target dosing is unknown, and unknowable from the prior art. We have discovered that in order to duplicate the bioavailability of triethylenetetramine in 300 mg triethylenetetramine dihydrochloride, about 701 mg of triethylenetetramine disuccinate is required. In order to duplicate the bioavailability of triethylenetetramine in 250 mg triethylenetetramine dihydrochloride, we discovered that about 584 mg of triethylenetetramine disuccinate is required. In order to duplicate the bioavailability of triethylenetetramine in 250 mg triethylenetetramine tetrahydrochloride (which is bioequivalent to the dihydrochloride salt), we discovered that about 350 mg of triethylenetetramine disuccinate is required.

Thus, we not only discovered that fixed doses comprising or consisting essentially of about 701 mg of triethylenetetramine disuccinate and about 584 mg of triethylenetetramine disuccinate, and about 350 mg of triethylenetetramine disuccinate are optimal for dosing this salt form, but that per day doses of about 2336 mg and 2804 mg of triethylenetetramine disuccinate are optimal for treatment of Wilson's disease and other copper disorders, based on 1000 mg/day and 1200 mg/day dosing respectively.

Using the above per day triethylenetetramine dihydrochloride pediatric and adult dosing ranges of 500-750 mg for children and 750-1250 mg (and up to 2000 mg/day) for adults, the per day triethylenetetramine disuccinate dose ranges are from about 1168 mg to about 1752 mg for children and from about 1752 mg to about 2920 mg (and up to 4672 mg/day) for adults. Doses are increased if the clinical response not adequate or free serum copper are persistently >20 mcg/dL, and long-term maintenance doses are reassessed every 6-12 months.

Another approved daily dose of trientine dihydrochloride is 1200-2400 mg/day in 2-4 divided doses for adults, and a lower dose, typically 600-1500 mg/day, depending on age and body weight, for children, also typically given in divided doses. Based on the discoveries herein, the superior triethylenetetramine disuccinate salt would be dosed at about 2803 mg/day to about 5606 mg/day for adults, and about 1402 mg/day to about 3504 mg/day, depending on age and body weight, for children, all typically given in divided doses.

Cuprior® (triethylenetetramine tetrahydrochloride) is also indicated for the treatment of Wilson's disease in adults, adolescents and children ≥5 years intolerant to D-penicillamine therapy and is sold as 150 mg tablets. The approved and recommended Cuprior® dosing regimen for adults is between 450 mg and 975 mg (3 to 6½ tablets) per day in 2 to 4 divided doses. The triethylenetetramine disuccinate dosing regimen for adults would be between about 1051 mg and about 2278 mg per day (typically using a 350-350.4 mg fixed dose, which corresponds to the 150 mg triethylenetetramine tetrahydrochloride tablet). The starting dose in pediatrics is lower than for adults and depends on age and body weight. In general, the Cuprior® dose for children is usually between 225 mg and 600 mg per day (1½ to 4 tablets) in 2 to 4 divided doses. The triethylenetetramine disuccinate dosing regimen for children would be between about 525 mg and about 1400 mg per day.

We further discovered that other fixed doses of triethylenetetramine disuccinate for optimal dosing and bioavailability are about 350 mg, about 400 mg, about 500 mg, about 600 mg and about 700 mg of triethylenetetramine disuccinate, including fixed doses of about 350.4 mg, 584 mg and about 701 mg of triethylenetetramine disuccinate. Exemplary effective amounts are described herein, and include doses in the range of from about 2300 mg per day to about 2800 mg per day given as multiple fixed doses of triethylenetetramine disuccinate comprising or consisting essentially of about 350 mg, 400 mg, about 500 mg, about 600 mg and/or about 700 mg, for example. Other fixed doses of triethylenetetramine disuccinate are given to equal about 1050 mg/day to about 2300 mg/day, about 1400 mg/day to about 3500 mg/day, about 2400 mg/day to about 3000 mg/day, and about 2800 mg/day to about 5600 mg/day.

By way of example, four 350 mg triethylenetetramine disuccinate capsules given BID would equal 2800 mg per day (roughly equivalent to 2804 mg per day, which is the triethylenetetramine disuccinate dose that we discovered is bioequivalent to the 1200 mg per day triethylenetetramine dihydrochloride administered in Europe for treating Wilson's Disease). Three 400 mg triethylenetetramine disuccinate capsules, for example, given BID would equal 2400 mg per day (roughly equivalent to 2337 mg per day, which is the triethylenetetramine disuccinate dose that we discovered is bioequivalent to the 1000 mg per day triethylenetetramine dihydrochloride administered in the United States for treating Wilson's Disease).

Three 500 mg triethylenetetramine disuccinate fixed dose tablets/capsules, etc., for example, given BID would equal 3000 mg per day, roughly equivalent to the 2804 mg per day triethylenetetramine disuccinate bioequivalent dose we discovered. Four 600 mg triethylenetetramine disuccinate fixed dose tablets/capsules, etc., for example, given BID equals 2400 mg per day, which is roughly equivalent to the 2337 mg per day triethylenetetramine disuccinate bioequivalent dose we discovered. Two 700 mg triethylenetetramine disuccinate fixed dose tablets/capsules, etc., for example, given BID would equal 2800 mg per day, which is roughly equivalent to the 2804 mg per day triethylenetetramine disuccinate bioequivalent dose.

Other convenient fixed dose amounts of triethylenetetramine disuccinate can be calculated and manufactured to provide daily bioequivalent doses, such as about 2804 mg per day and about 2337 mg per day. For example, five 280 mg triethylenetetramine disuccinate doses given BID can be used to provide 2800 mg per day. Also, by way of example, four 290 mg triethylenetetramine disuccinate doses given BID can be used to provide 2320 mg per day.

Fixed doses of about 350 mg, about 584 mg and about 701 mg of triethylenetetramine disuccinate may also be given as two doses BID to equal per day doses of about 1400 mg, about 2336 mg and about 2804 mg of triethylenetetramine disuccinate, respectively. These and other fixed doses and total per day dose amounts described herein may be used to treat Wilson's disease and other copper disorders, including those described or referenced herein.

In general, the dosing is between about 2.336 and 2.337 mg of triethylenetetramine disuccinate for every milligram of triethylenetetramine dihydrochloride or triethylenetetramine tetrahydrochloride.

By way of example, we have also discovered that 2804 mg triethylenetetramine disuccinate per day, given as two 700 mg capsules administered twice daily, for example, would be expected to produce a significant cupruresis effect throughout the dosing interval with minimal side effects and negligible adverse effects on serum copper levels or other laboratory test parameters for treatment of heart disease, including, for example, heart disease in type 2 diabetic patients, in whom cardiomyopathy (e.g., elevated left ventricular mass) may also be treated with these dose amounts. Six months of treatment of elevated left ventricular mass with triethylenetetramine disuccinate dosed as described to provide about 2800 mg per day will cause elevated left ventricular mass to decline significantly toward normal.

We further discovered that fixed doses of triethylenetetramine disuccinate for optimal dosing and bioavailability given as multiple fixed doses of triethylenetetramine disuccinate comprising or consisting essentially of about 350 mg, 400 mg, about 500 mg, about 584, about 600 mg and/or about 700 or 701 mg will be useful for treating a copper-related disease, disorder or condition, as described herein.

In one aspect, the invention relates to newly discovered fixed dose amounts of triethylenetetramine disuccinate, formulations thereof, and their use for the treatment, prevention or amelioration of diseases, conditions and disorders treatable with copper chelators.

In certain embodiments, triethylenetetramine disuccinate is administered at an initial dose (or loading dose) followed by a maintenance dose, wherein the loading dose is about or at least 1.5 times greater, about or at least 2 times greater, about or at least 2.5 times greater, or about or at least 3 times greater than the maintenance dose. The maintenance dose may be, for example, about 350 mg, 400 mg, about 500 mg, about 584 mg, about 600 mg and/or about 700 or 701 mg, from 1-4 times per day. In one embodiment, the loading dose is administered once, twice, three, four, or five times before the first maintenance dose, and may be given once, twice, three times or four times a day.

Thus, by way of example, in one embodiment, for a 2337 mg per day triethylenetetramine disuccinate loading dose regimen, triethylenetetramine disuccinate is administered at a daily loading dose (which can be provided in one or several dosages throughout the day) of at least about 3505 mg (1.5×), at least about 4674 mg (2×), at least about 5842 mg (2.5×), or at least about 7001 mg (3×). In one embodiment, the triethylenetetramine disuccinate loading dose is administered in two doses a day, and optionally over 1, 2, 3, 4 or 5 or more days. Other triethylenetetramine disuccinate loading doses are calculated accordingly, based on triethylenetetramine disuccinate maintenance doses given daily or in other frequencies, such as, for example, 2804 or other maintenance doses given daily.

In one embodiment, the triethylenetetramine disuccinate fixed described herein doses are administered twice per day (BID) to provide the desired per day dosing. In another embodiment, the triethylenetetramine disuccinate fixed doses are administered three times per day (TID) to provide desired per day dosing. In a still further embodiment, the triethylenetetramine disuccinate fixed doses are administered four times per day (QID) to provide desired per day dosing.

Importantly, the crystalline anhydrous form of the triethylenetetramine disuccinate article of manufacture described herein has a shelf-life of at least about 12 months (and up to five years) at room temperature, without significant degradation of the triethylenetetramine disuccinate API and remains within impurity specifications for the triethylenetetramine disuccinate drug substance. In one embodiment, the term “without significant degradation” means that the purity of the triethylenetetramine disuccinate is at least about 98.5% with no degradation product above about 0.5% and no new, unidentified impurities above about 0.1% for at least about 12 months.

### Definitions

Copper (II) referred to herein is also known as or Cu⁺² or copper⁺², or as “cupric” (the copper⁺² cation).

The term “comprising,” which is synonymous with “including,” “containing,” or “characterized by,” is inclusive or open-ended and does not exclude additional, unrecited elements or ingredients from the medicament (or steps, in the case of a method). The phrase “consisting of” excludes any element, step, or ingredient not specified in the medicament (or steps, in the case of a method). The phrase “consisting essentially of” refers to the specified materials and those that do not materially affect the basic and novel characteristics of the medicament (or steps, in the case of a method). The basic and novel characteristics of the inventions are described throughout the specification, and include the ability of compounds, compositions and methods of the invention to reduce copper levels, to reduce total copper, to reduce copper values, to reduce copper(II), and/or to chelate copper(II). The basic and novel characteristics of the inventions also include the ability of compounds, compositions and methods of the invention to provide a clinically relevant change in a copper-related disease, disorder or condition, or a symptom thereof. In another aspect of one of the methods of the inventions, the basic and novel characteristics of other compositions and methods of the invention include the ability to reduce inflammation and/or combat vessel leak. This concept incorporates, for example, the ability to reduce or eliminate the deleterious effects of excessive fluid migration through the walls of the vasculature or into extracellular matrices and diseases that are initiated by or exacerbated by such processes. Aside from the use of the terms “comprising” and “consisting essentially of” in the appended claims, the term “comprising” as used in the Specification may be substituted for “consisting essentially of,” and vice versa. Thus, by way of example, the phase “a composition comprising X” as used in the Specification may be written in the claims as either “a composition comprising X” or “a composition consisting essentially of X.”

As used herein, the term “subject” or the like, including “individual,” and “patient”, all of which may be used interchangeably herein, refers to any mammal, including humans. The preferred mammal herein is a human, including adults, children, including those with Wilson's Disease, heart failure, cardiomyopathy, left ventricular hypertrophy, diabetes or cancer, by way of example. In certain embodiments, the subject, individual or patient is a human.

As used herein, “mammal” has its usual meaning and includes primates (e.g., humans and nonhumans primates), experimental animals (e.g., rodents such as mice and rats), farm animals (such as cows, hogs, minks, sheep and horses), and domestic animals (such as dogs and cats). In one aspect of the invention, fixed doses of triethylenetetramine disuccinate are added to animal feed or water. The invention includes articles of manufacture comprising fixed doses of triethylenetetramine disuccinate in animal feed or water.

As used herein the terms “subjecting the patient” or “administering to” includes any active or passive mode of ensuring the in vivo presence of triethylenetetramine disuccinate. Preferably the mode of administration is oral. However, all other modes of administration (particularly parenteral, e.g., intravenous, intramuscular, etc.) are contemplated.

The term “treating a copper-related disease, disorder or condition” or the like, refers to preventing, slowing, reducing, decreasing, stopping and/or reversing a disease, disorder or condition characterized by pathological, excess or unwanted copper, or a disease, disorder or condition treatable with a copper(II) chelator, or one or more symptoms thereof, including, for example, Wilson's Disease, heart failure, diabetic cardiomyopathy, left ventricular hypertrophy, diabetes mellitus, Alzheimer's Disease, Parkinson's Disease, idiopathic pulmonary fibrosis, and cancer. The triethylenetetramine disuccinate doses and methods of treatment described herein may be used to treat copper-related diseases, disorders or conditions.

“Treating copper excess” refers to preventing, slowing, reducing, decreasing, stopping and/or reversing, in whole or in part, pathological, excess or unwanted copper in a subject, and/or to treating one or more symptoms of excess or unwanted copper. The triethylenetetramine disuccinate doses and methods of treatment described herein may be used to treat copper excess.

The term “preventing” means preventing in whole or in part or ameliorating or controlling. Thus, preventing a disease, disorder or condition means preventing in whole or in part, or ameliorating or controlling the disease, disorder or condition. The triethylenetetramine disuccinate doses and methods of treatment described herein may be used to prevent copper excess, and copper-related diseases, disorders or conditions.

As used herein, the terms “effective amount” or “therapeutically effective amount” refer to an amount of triethylenetetramine disuccinate described herein. Exemplary effective amounts are described herein, and include doses in the range of from about 2400 mg per day to about 3000 mg per day comprising or consisting essentially of fixed doses of triethylenetetramine disuccinate. In one aspect, the effective amount of triethylenetetramine disuccinate is at least about 95% pure, at least about 99% pure, or 100% pure. In another aspect, the effective amount of triethylenetetramine disuccinate is a crystalline form of triethylenetetramine disuccinate. In yet another aspect of this method, the effective amount of triethylenetetramine disuccinate is a triethylenetetramine disuccinate anhydrate. In still another aspect of the method the effective amount of the triethylenetetramine disuccinate is in the form of a fixed dose tablet or capsule. In one preferred embodiment, the effective fixed dose of triethylenetetramine disuccinate is about 400 mg, about 500 mg, about 600 mg or about 700 mg. A fixed dose of 350 mg is also described.

Thus, in one aspect, “effective amount” refers to an amount effective, at dosages and for periods of time necessary, to achieve the desired therapeutic or prophylactic result. For example, and not by way of limitation, an “effective amount” can refer to an amount of a triethylenetetramine disuccinate disclosed herein that is able to treat the signs and/or symptoms of the copper-related disease, disorder or condition, or otherwise treat copper excess. In one embodiment, the effectiveness of the amount is evaluated by determining the response of the subject and/or the amount copper in the urine or plasma of a subject following the dosing of triethylenetetramine disuccinate as disclosed herein. Preferably, the effective amount maintains normal copper levels, or maintains a subject's copper levels within at least about 70% of normal, or within other levels described herein.

As used herein, “prophylactically effective amount” refers to an amount effective, at dosages and for periods of time necessary, to achieve a desired prophylactic result. Typically, but not necessarily, since a prophylactic fixed dose of triethylenetetramine disuccinate is used in subjects prior to or at an earlier stage of a copper-related disease, disorder or condition, the prophylactically effective amount may be less than the therapeutically effective amount. Prophylactic doses may also serve as maintenance doses once a copper-related disease, disorder or condition has been brought under control with, for example, an initial, bolus or loading dose or doses, all as described herein, for example.

By “pharmaceutically acceptable” it is meant, for example, a carrier, diluent or excipient that is compatible with the other ingredients of the formulation and generally safe for administration to a recipient thereof or that does not cause an undesired adverse physical reaction upon administration. A “pharmaceutically acceptable carrier,” as used herein, refers to an ingredient in a pharmaceutical formulation, other than an active ingredient, which can be safely administered to a subject. A pharmaceutically acceptable carrier includes, but is not limited to, a buffer, excipient, stabilizer, or preservative. Pharmaceutically acceptable diluents, carriers and/or excipients include substances that are useful in preparing a pharmaceutical composition, may be co-administered with compounds described herein while allowing them to perform its intended functions, and are generally safe, non-toxic and neither biologically nor otherwise undesirable. Pharmaceutically acceptable diluents, carriers and/or excipients include those suitable for veterinary use as well as human pharmaceutical use.

The term “pharmaceutical formulation” refers to a preparation which is in such form as to permit the biological activity of the triethylenetetramine disuccinate contained therein to be effective, and which does not contain additional components that are unacceptably toxic to a subject to which the formulation would be administered. Pharmaceutical formulations of the invention comprise a fixed dose of triethylenetetramine disuccinate as disclosed herein and a pharmaceutically acceptable carrier.

“Copper chelating agents” bind or modify copper, including those that selectively bind to or modify copper (I) or copper(II) values and are used to normalize blood and/or tissue copper levels and to prevent unwanted copper accumulation. Copper chelating agents include prodrugs thereof. Other agents that normalize copper values, and other agents that selectively bind to or modify copper(II), whether now known or later developed, are included within this definition.

A “copper sequestering agent” or “copper-depriving agent” is an agent that can bind to and/or suppress the ability of copper in any or all of its various forms. Copper-depriving agents include chelators, agents that reduce total copper, agents that reduce copper values, agents that reduce the amount of intracellular copper available, including those described herein. Copper-depriving agents also include copper-modifying agents, i.e., agents used to reduce copper by modifying copper content in the body, including intracellular content, or by modifying copper availability. It is understood that copper is an essential intracellular nutrient, and thus the invention includes methods to reduce intracellular copper content while maintaining safe patient copper levels. Copper-depriving agents include copper-removing agents, i.e., agents that remove copper from the body and/or from inside cells.

As used herein, the terms “treatment” or “treating” of the signs and/or symptoms of a copper-related disease, disorder or condition, in a mammal, means, where the context allows, (i) preventing the condition or disease, that is, avoiding one or more clinical symptoms of the disease; (ii) inhibiting the condition or disease, that is, arresting the development or progression of one or more clinical symptoms; and/or (iii) relieving the condition or disease, that is, causing the regression of one or more clinical symptoms. Thus, “treatment” (and grammatical variations thereof such as “treat” or “treating”) normally refers to clinical intervention in an attempt to alter the natural course of the individual, tissue or cell being treated, and can be performed either for prophylaxis or during the course of clinical pathology. The term does not necessarily imply that a subject is treated until total recovery. Accordingly, “treatment” includes reducing, alleviating or ameliorating the symptoms or severity of a copper-related disease, disorder or condition, or preventing or otherwise reducing the risk of developing a copper-related disease, disorder or condition. It may also include maintaining or promoting a complete or partial state of remission of a copper-related disease, disorder or condition. The triethylenetetramine disuccinate doses described herein are used for treatment.

As used herein “associated with” simply means both circumstances exist and should not be interpreted as meaning one necessarily is causally linked to the other.

The term “chelatable copper” includes copper in any of its chelatable forms that can be bound by triethylenetetramine disuccinate, such copper(II). Accordingly, the term “copper values” (for example, elemental, salts, etc.) means copper in any appropriate form in the body available for such chelation by triethylenetetramine disuccinate (for example, in extracellular tissue and possibly bound to cell exteriors and/or collagen as opposed to intracellular tissue). Certain methods and compositions of the invention may be used to bind chelatable copper, for example, chelatable copper(II) while maintaining normal or near-normal copper values (e.g., within about 70-75% of normal, for example, or other copper values amount not detrimental to the subject).

The triethylenetetramine disuccinate doses described and claimed selectively bind to or modify copper(II) values and are used to normalize blood and/or tissue copper levels and to prevent unwanted copper accumulation, and administered to a subject with a disease, disorder or condition treatable by a copper chelator.

Triethylenetetramine disuccinate include prodrugs thereof, with doses modified to account for the molecular weight of the “pro-” portion of the triethylenetetramine disuccinate prodrug.

The doses of triethylenetetramine disuccinate disclosed herein may be administered alone or in combination with one or more additional ingredients and may be formulated into pharmaceutical compositions including one or more pharmaceutically acceptable excipients, diluents and/or carriers. In certain embodiments, the invention provides a combination product comprising (a) a dose of triethylenetetramine disuccinate, and (b) one or more anti-inflammatory agents and/or anti-vessel-leak agents, wherein the components (a) and (b) are adapted for administration simultaneously or sequentially. In a particular embodiment of the invention, a combination product in accordance with the invention is used in a manner such that at least one of the components is administered while the other component is still having an effect on the subject being treated. The dose of triethylenetetramine disuccinate and/or anti-inflammatory agents and/or anti-vessel leak agents may be contained in the same or one or more different containers and administered separately, or mixed together, in any combination, and administered concurrently. Preferably, both or all three of the triethylenetetramine disuccinate and/or anti-inflammatory agent and/or anti-vessel leak agent are combined in a capsule for oral administration.

In another embodiment, the fixed dose of triethylenetetramine disuccinate is used in combination with an inhibitor of the metabolism of triethylenetetramine. This is contemplated to include (although not to be in any way limited by this description), inhibitors of the enzymes N-acetylaminotransferase and/or spermine/spermidine N-acetylaminotransferase (SSAT1 and/or SSAT2).

Triethylenetetramine is understood to operate as a strong chelator of copper(II) ions. Thus, treatments for Wilson's Disease, by way of example, are believed to operate at least in part by enhancing the elimination of copper(II) ions from the body. Triethylenetetramine may also exert a dual positive effect in treatment, however, by decreasing copper absorption from the gut. For instance, inhibiting the action of copper-dependent enzymes can be optimized by achieving effective concentrations of triethylenetetramine either extracellularly or intracellularly so as to competitively inhibit the action of these enzymes. Under these conditions, enhanced elimination of copper would offer additional therapeutic utility.

Syprine® is known to be characterized by very poor intestinal absorption (oral bioavailability substantially less than 10%). This is in part explained by its multiple ionizable (basic) amine groups. The enhanced absorption of triethylenetetramine would allow for substantial reduction of doses, likely with improved side effects profile and perhaps even better efficacy.

The rapid metabolism of triethylenetetramine by N-acetylaminotransferase or by spermine/spermidine N-acetylaminotransferase (SSAT 1 and/or SSAT2) enzymes in the liver—or wherever such enzymes are expressed in sufficient concentrations—is a limiting factor that contributes to the high doses required for Syprine® and its other salt forms. Thus, co-administration of trientine or its various salt forms including triethylenetetramine disuccinate with an inhibitor of these enzymes can be beneficial to enhanced bioavailability and lowering of the dose. Thus, triethylenetetramine disuccinate (or triethylenetetramine dihydrochloride or triethylenetetramine tetrahydrochloride) may be formulated or co-administered with inhibitors of N-acetylaminotransferase and/or spermine/spermidine N-acetylaminotransferase (SSAT1 and/or SSAT2). Such agents include molecules such as acetaminophen, di-allylsulfide and caspofungin. In another aspect, the fixed dose of triethylenetetramine disuccinate is used in combination with an inhibitor of spermidine-spermine-N(1)-acetyltransferase (SSAT1 and/or SSAT2).

Such combination products may be manufactured in accordance with the methods and principles provided herein and those known in the art. Also provided is combination product used in a method as herein described.

For separate or common administration, the fixed dose triethylenetetramine disuccinate formulation may be prepared to provide for rapid or slow release; immediate, delayed, timed, or sustained release; or a combination thereof. Formulations may be in the form of liquids, solutions, suspensions, emulsions, elixirs, syrups, electuaries, drops (including but not limited to eye drops), tablets, granules, powders, lozenges, pastilles, capsules, gels, ointments, creams, lotions, oils, foams, sprays, mists, or aerosols. For instance, a stomach-retentive or a mucoadhesive formulation of triethylenetetramine disuccinate can enhance or to extend the absorption of this therapeutic article in the GI tract. A delayed release form of the triethylenetetramine disuccinate will serve to avoid metabolism, prolong and increase absorption, and increase bioavailability by releasing the drug after it passes the stomach. Various different means are available to accomplish these modified release formulations. Such technologies are well-known to one of skill in the art, with specific techniques and excipients selected to address issues or challenges posed by the ADME profile of the article in question.

Mucoadhesive formulations contains specific polymers that adhere to the epithelial lining at the site where they are hydrated. Thus, a drug that is released, for example, in the duodenum after transit through the stomach will adhere to the walls of the GI tract, causing extended and preferential drug release and absorption from this site. Buccal, corneal, respiratory, and vaginal tissues are also lined with mucosal tissues and are thus targets for such formulations. The muco-adhesive properties of most polymers increase with molecular weight, thus MWs in the range of 200,000-700,000, for example, are found to correlate with enhanced muco-adhesion for polyoxyethylene polymers and copolymer. Viscosity, pore size, and the degree of cross linking are other factors that are considered in the selection of muco-adhesive polymers. Hydrogen bonding, flexibility, degree of hydration, and swell are also important factors in drug delivery from muco-adhesive polymers. In addition to polyoxyethylene/polyvinyl alcohol, materials composed of polymeric acrylic and methacrylic esters, and hydroxylated methacrylic polymers are useful for this purpose. Chitosan, cyanoacrylates, hyaluronic acid, hydroxypropyl celluloses, gellan, polycarbopol, and sodium carboxymethylcelluloses are other related polymers have been used in muco-adhesive formulations. Nasal muco-adhesive formulations are developed with attention to the specific properties of such tissues. Nasal delivery system include copolymers of methyl vinyl ether, (hydroxypropyl)methylcellulose (HPMC), sodium carboxymethylcellulose, carbopol-934P and Eudragit RL-10. Mucin, gelatin, polycarbophil, and poloxamer are examples of polymers used for vaginal or rectal muco-adhesive formulations. Oral delivery systems for GI muco-adhesive systems are represented by chitosan, polyacrylic acid, alginate, polymethacrylic acid and sodium carboxymethyl cellulose. Muco-adhesive fixed dose triethylenetetramine disuccinate formulations can be prepared using such compounds.

Stomach retentive formulations are generally designed for drugs that have an optimal window of absorption in the stomach and proximal intestine. Hydrodynamically balanced systems, floating microspheres, gas-generating tablets, formulations that swell to prevent passage from the stomach, and formulations that adhere to the walls of the stomach are examples of such formulations. “Plug” systems that expand to a size that they cannot readily pass the pyloric sphincter are one example of stomach retentive formulations. Low-density (floating) or gas-generating (carbon dioxide) formulations are retained for extended periods of time; such techniques may be used in combination to optimize such performance. Muco-adhesive polymers are also often used to design such an effect into a formulation. Sodium alginate in combination with sodium carbonate or sodium bicarbonate can result in a “rafting” effect such that formulations are retained in the stomach based on buoyancy in the stomach liquid. Stomach-retentive fixed dose triethylenetetramine disuccinate formulations can be prepared using these methods and compounds.

Preferred copper chelating agents used in methods of the invention are the fixed doses described herein, and the total administered amounts of triethylenetetramine disuccinate using those fixed doses.

In one aspect, the fixed dose of triethylenetetramine disuccinate is substantially pure, including at least about 90% pure, at least about 95% pure and 100% pure. In one aspect the triethylenetetramine disuccinate is triethylenetetramine disuccinate anhydrate. In one aspect the triethylenetetramine disuccinate is crystalline form of triethylenetetramine disuccinate or triethylenetetramine disuccinate anhydrate.

In another aspect of the invention the pharmaceutically acceptable salt is a polymorph of triethylenetetramine disuccinate. Triethylenetetramine disuccinate polymorphs are described in, for example, U.S. Pat. No. 8,067,641. In one aspect, the fixed dose comprises a polymorph of a triethylenetetramine disuccinate wherein the polymorph is a crystal having the structure defined by the co-ordinates of Table 3B found in U.S. Pat. No. 8,067,641. In another aspect of the invention fixed dose comprises a polymorph of triethylenetetramine disuccinate wherein the polymorph is a crystal having the structure defined by the co-ordinates of Table 3C found in U.S. Pat. No. 8,067,641. In other aspects of the invention, the fixed triethylenetetramine disuccinate dose consists essentially of a triethylenetetramine disuccinate polymorph having a crystal having the structure defined by the co-ordinates of Table 3B in U.S. Pat. No. 8,067,641, or consists essentially of a crystalline triethylenetetramine disuccinate polymorph having the structure defined by the co-ordinates of Table 3C in U.S. Pat. No. 8,067,641.

### Fixed Dose Amounts and Daily or Other Periodic Dosing

Effective fixed triethylenetetramine disuccinate dose amounts are about 400 mg, about 500 mg, about 600 mg or about 700 mg. A fixed dose of 350 mg is also provided. The fixed dose amounts are used, for example, to administer triethylenetetramine disuccinate doses in the range of from about 2400 mg per day to about 3000 mg per day, or other period of time. In one aspect, the effective amount of triethylenetetramine disuccinate is at least about 95% pure, at least about 99% pure, or 100% pure. In another aspect, the effective amount of triethylenetetramine disuccinate is a crystalline form of triethylenetetramine disuccinate. In yet another aspect of this method, the effective amount of triethylenetetramine disuccinate is a triethylenetetramine disuccinate anhydrate. In still another aspect of the method the effective amount of the triethylenetetramine disuccinate is in the form of a fixed dose tablet or capsule. The total dosage may be given in single or divided dosage units (e.g., BID, TID), and preferably maintain normal urine and/or plasma copper levels in a subject, or levels that do not fall below about 70% to 75% of normal. In one preferred embodiment, the fixed doses are administered BID.

### Other Indications

In some embodiments, the invention provides a cosmetic composition comprising a cosmetically effective quantity of a copper sequestering agent or copper-depriving agent, e.g., a copper chelator, such as a copper(II) chelator, for example, triethylenetetramine disuccinate. In some embodiments, the invention provides methods for preventing or slowing hair loss or promoting hair development by administration of a composition comprising or consisting essentially of a copper sequestering agent or copper-depriving agent. In some embodiments, the invention provides methods for preventing, reducing or eliminating liver spots or promoting normal skin development by administration of a composition comprising or consisting essentially of a copper sequestering agent or copper-depriving agent. In some embodiments, the invention provides methods for preventing, reducing or eliminating cellulite or promoting normal skin development by administration of a composition comprising or consisting essentially of a copper sequestering agent or copper-depriving agent. In some embodiments, the copper sequestering agent or copper-depriving agent is a copper chelator. In some embodiments, the copper chelator is a copper(II) chelator, for example, a triethylenetetramine. In some embodiments, the triethylenetetramine is triethylenetetramine disuccinate. In some embodiments, the copper sequestering agent or copper-depriving agent for preventing or slowing hair loss or promoting hair development, for preventing, reducing or eliminating liver spots or cellulite or promoting normal skin development is administered as a topical pharmaceutical formulation. In some embodiments, the topical formulation is provided in the form of a paste, ointment, oil, cream, lotion, foam, gel, tincture, powder, spray or patch. In some embodiments, the copper sequestering agent or copper-depriving agent for preventing or slowing hair loss or promoting hair development, for preventing, reducing or eliminating liver spots or cellulite or promoting normal skin development is administered by microneedles, or by adhesive patches, non-adhesive patches, occlusive patches or microelectric patches. In some embodiments, the copper sequestering agent or copper-depriving agent for preventing or slowing hair loss or promoting hair development, for preventing, reducing or eliminating liver spots or cellulite or promoting normal skin development is administered enterally (e.g., orally, sublingually, buccally, rectally, etc.), parenterally (e.g., intravenously, intramuscularly, subcutaneously, etc.), intranasally, by transdermal administration, ophthalmic administration, by inhalation, etc. In some embodiments, the copper sequestering agent or copper-depriving agent for preventing or slowing hair loss or promoting hair development, for preventing, reducing or eliminating liver spots or cellulite or promoting normal skin development is administered using a delayed or sustained release system. The amount of compound contained in the sustained release system will depend, for example, on where the composition is to be administered, the kinetics and duration of the release of the compound of the invention, as well as the nature of the condition, disorder and/or disease to be treated and/or cared for. A person skilled in the art knows the different means by which the cosmetic or pharmaceutical compositions which contain the compounds of the invention can be administered.

In some embodiments, the area undergoing treatment to prevent or slow hair loss or promote hair development, to prevent, reduce or eliminate liver spots or cellulite or otherwise promote normal skin development is pre-treated in order to facilitate transport of the copper sequestering agent or copper-depriving agent to the desired region of the skin and/or skin-associated (e.g., epidermis, dermis, basal layer, and/or hair follicle) area(s). For example, a skin region to be treated can be treated by dermabrasion techniques (such as application of sugar crystals, cellulosic plant matter, frozen CO₂, polymeric beads, and/or silica granules), by manual application, etc. Similarly, a skin region to be treated with a copper sequestering agent or copper-depriving agent can be prepared by application of chemical agents that reduce the thickness or increase the permeability of the stratum corneum, such as surfactants and/or chemical reductants. In some embodiments, skin and/or skin-associated areas can be prepared by the application of pulsed laser light and/or acoustic energy (e.g., via ultrasound) prior to or with application of a copper sequestering agent or copper-depriving agent. Similarly, skin can be prepared stripping techniques (such as the application and subsequent removal of an adhesive film, tape, or wax) prior to application of a copper sequestering agent or copper-depriving agent. Skin can be treated with a copper sequestering agent or copper-depriving agent containing composition prior to, during, and/or following such skin preparative treatments. For example, skin preparative treatments as described above can be applied prior to application of a defensin-containing composition. Alternatively, skin preparative treatments as described above can be applied following application of a defensin-containing preparation to the skin. Copper sequestering agents and copper-depriving agents can be applied in a continuous or discontinuous fashion.

### Manufacture

Triethylenetetramine disuccinate suitable for use in the present invention may be obtained from known manufacturing sources or synthesized using methods known in the art. Manufacturing methods are described in U.S. Pat. No. 9,556,123, for example, which describes the synthesis of triethylenetetramines and useful intermediates in their production. U.S. Pat. No. 8,067,641 describes methods for the synthesis of substantially pure triethylenetetramine disuccinate, substantially pure triethylenetetramine disuccinate anhydrate, and triethylenetetramine disuccinate polymorphs.

Other copper sequestering agents and copper-depriving agents, including other copper chelators, as well as composition and preparations can be acquired from various manufacturers or manufacturing sources or made according to methods in the art.

### Pharmaceutical Preparations

Also provided are pharmaceutical preparations that include a fixed dose of triethylenetetramine disuccinate present in a pharmaceutically acceptable vehicle. The term “pharmaceutically acceptable” has the meaning set forth above and includes those vehicles approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the invention is formulated for administration to a mammal.

In one aspect, the present disclosure provides pharmaceutical preparations wherein the fixed dose triethylenetetramine disuccinate (alone or together with another active ingredient) is prepared by combining it (or them) with one or more pharmaceutically acceptable diluents, carriers, adjuvants, and the like in a manner known to those skilled in the art of pharmaceutical formulation. The fixed dosage form can be prepared by combining it with one or more pharmaceutically acceptable diluents, carriers, adjuvants, and the like in a manner known to those skilled in the art of pharmaceutical formulation.

The choice of excipient will be determined in part by the active ingredient, as well as by the particular method used to administer the composition. Accordingly, there is a wide variety of suitable formulations of the pharmaceutical composition of the present invention.

Dosage forms useful herein include any appropriate dosage form known in the art to be suitable for pharmaceutical formulation of compounds suitable for administration to mammals particularly humans, particularly (although not solely) those suitable for stabilization in solutions, tablets or capsules comprising therapeutic compounds for administration to humans.

Compositions may take the form of any standard known dosage form, including those mentioned above, and including tablets, pills, capsules, semisolids, powders, sustained release formulation, solutions, suspensions, elixirs, aerosols, liquids for injection, transdermal delivery devices (for example, a transdermal patch), or any other appropriate compositions. Persons of ordinary skill in the art to which the invention relates will appreciate the most appropriate dosage form having regard to the nature of the condition to be treated and the active agent to be used without any undue experimentation. Fixed triethylenetetramine disuccinate doses and dose ranges are described herein. It should be appreciated that one or more of the other active agents (e.g., an anti-inflammatory agent) may be formulated into a single composition with the triethylenetetramine disuccinate fixed dose. In certain embodiments, preferred dosage forms include an injectable solution, a topical formulation in a transdermal patch, and an oral formulation. The dosage forms of the invention include any appropriate dosage form now known or later discovered in the art to be suitable for pharmaceutical formulation of compounds suitable for administration to humans.

One example is oral delivery forms of tablet, capsule, lozenge, or the like, or any liquid form, capable of protecting the compound from degradation prior to eliciting an effect, for example, in the alimentary canal if an oral dosage form.

Particular formulations of the invention are in a solid form, particularly tablets or capsules for oral administration.

Slow- or modified-release preparations of triethylenetetramine disuccinate in tablets or capsules are preferred.

In addition to standard diluents, carriers and/or excipients, a composition in accordance with the invention may be formulated with one or more additional constituents, or in such a manner, so as to enhance activity or bioavailability, help protect the integrity or increase the half-life or shelf life thereof, enable slow release upon administration to a subject, or provide other desirable benefits, for example. For example, slow-release vehicles include macromers, poly(ethylene glycol), hyaluronic acid, poly(vinylpyrrolidone), or a hydrogel so as to allow for sustained release of the product from the matrix over time. By way of further example, the compositions may also include preserving agents, solubilizing agents, stabilizing agents, wetting agents, emulsifying agents, sweetening agents, coloring agents, flavoring agents, coating agents, buffers and the like. Those of skill in the art to which the invention relates will readily identify further additives that may be desirable for a particular purpose.

The fixed triethylenetetramine disuccinate doses of the invention may be administered by a sustained-release system. Suitable examples of sustained-release compositions include semi-permeable polymer matrices in the form of shaped articles, e.g., films, or microcapsules. Sustained-release matrices include polylactides (U.S. Pat. No. 3,773,919; EP 58,481), copolymers of L-glutamic acid and gamma-ethyl-L-glutamate, poly(2-hydroxyethyl methacrylate), ethylene vinyl acetate, or poly-D-(−)-3-hydroxybutyric acid (EP 133,988). Sustained-release compositions also include a liposomally entrapped (e.g., encapsulated) compound. Liposomes containing copper chelating agents (alone or together with an antiviral and/or anti-inflammatory agent) may be prepared by known methods, including, for example, those described in: DE 3,218,121; EP 52,322; EP 36,676; EP 88,046; EP 143,949; EP 142,641; Japanese Pat. Appln. 83-118008; U.S. Pat. Nos. 4,485,045 and 4,544,545; and EP 102,324. Ordinarily, the liposomes may be used to encapsulate the triethylenetetramine disuccinate and are of the small (from or about 200 to 800 Angstroms) unilamellar type in which the lipid content is greater than about 30 mole percent cholesterol, the selected proportion being adjusted for the most efficacious therapy. Slow release delivery using PGLA nano- or microparticles, or in situ ion activated gelling systems may also be used, for example.

Another objective of this invention is to provide formulations of triethylenetetramine disuccinate that are superior in their pharmacokinetic profile to earlier formulations. By superior it is understood that increasing the absorption, distribution, slowing metabolism, or elimination of triethylenetetramine may result in enhanced therapeutic efficacy, or equivalent efficacy at a lower dose. It is further understood that any or all of these same formulations may be practiced with different salt forms or the free amine base of triethylenetetramine so as to alter or improve their pharmacokinetic profiles as well, and so as to provide an improved or more suitable product for the intended use. Such objectives can variously be achieved by modifying the formulations of triethylenetetramine disuccinate or other salts of triethylenetetramine to achieve the desired outcome.

In one such instance, the desired salt form may be formulated using a gastro-retentive dose form (GRDF). Such delivery forms are formulated with the intent of prolonging gastric retention time, and thus enhancing absorption. Such strategies may employ for instance: 1) passage-delaying agents; 2) large single-unit dosage forms; 3) bioadhesive drug delivery systems; 4) heavy pellets; and 5) buoyant forms. Polymers such a Carbopol, chitosan, sodium alginate, HPMC, polyacrylic acids, polyethylene glycol and modified forms of these polymers are variously used to achieve gastric retention, as a few examples among others.

In a second instance of modified dosage forms for non-immediate release, the product is formulated to delay the release of the drug until after the dosage form exits the stomach. In a delayed release form, the release profile is similar or equal to that of an immediate release form, but the actual release of the drug is delayed by, e.g., enteric coating so that the active ingredient is not release from the dosage form granulation until after transit through the stomach is complete. Enteric coating as one example of this strategy is accomplished by using for instance, (meth)acrylic polymers which do not dissolve in aqueous medium until the pH is above 5.5, thus achieving a dosage form that transits the stomach without releasing the active ingredient.

Extended-release dosage forms are distinct from delayed release in that the release profile of the drug is extended beyond that of an immediate release product. Mechanisms for extended release include delayed dissolution, diffusion, delivery from an intact dosage form by osmotic pressure, maintaining a hydrologic or hydrodynamic balance, and ion exchange. A traditional means of obtaining extended-release delivery is to formulate in a matrix of a non-ionic cellulosic ether (such as HPMC; cf. U.S. Pat. No. 8,865,778B2) in the presence of a selected amount of non-crosslinked swelling agent such as carboxymethyl starch or sodium starch glycolate. Other approaches to achieving the same result are known. For instance, a drug delivery formulation core that contains an osmotic agent and a water-swellable polymer is readily used as a driving force to deliver a drug in a controlled, extended manner.

Therapeutic formulations for use in the methods and preparation of the compositions of the present invention can be prepared by any methods well known in the art of pharmacy. See, for example, Gilman et al. (eds.) GOODMAN AND GILMAN'S: THE PHARMACOLOGICAL BASES OF THERAPEUTICS (8th ed.) Pergamon Press (1990); and Remington, THE SCIENCE OF PRACTICE AND PHARMACY, 20th Edition. (2001) Mack Publishing Co., Easton, Pa.; Avis et al. (eds.) (1993) PHARMACEUTICAL DOSAGE FORMS: PARENTERAL MEDICATIONS Dekker, N.Y.; Lieberman et al. (eds.) (1990) PHARMACEUTICAL DOSAGE FORMS: TABLETS Dekker, N.Y.; and Lieberman et al. (eds.) (1990) PHARMACEUTICAL DOSAGE FORMS: DISPERSE SYSTEMS Dekker, N.Y. Compositions may also be formulated in accordance with standard techniques as may be found in such standard references as Gennaro A R: Remington: The Science and Practice of Pharmacy, 20.sup.th ed., Lippincott, Williams & Wilkins, 2000, for example.

Particular formulations of the invention are in a form for nasal administration, e.g., nanoemulsion. Other formulations of the invention are in the form of a transdermal patch.

Other formulations of the invention consist essentially of a fixed dose of triethylenetetramine disuccinate in an amount described herein. Preferred is a formulation consisting essentially of a fixed dose of substantially pure triethylenetetramine disuccinate anhydrate, i.e., at least about 90% pure, at least about 95% pure, or 100% pure.

### Articles of Manufacture/Kits

The invention also includes an article of manufacture, or “kit”, containing materials useful for treating a subject for a copper-related disease, disorder or condition is provided. The kit comprises a container comprising or consisting essentially of a fixed dose of triethylenetetramine disuccinate, preferably substantially pure triethylenetetramine disuccinate anhydrate. The kit may further comprise a label or package insert, on or associated with the container (or noted to be available online or in the cloud, or in a flash drive or another storage mechanism). The term “package insert” is used to refer to instructions customarily included in commercial packages of therapeutic products (or available online), that contain information about the indications, usage, dosing, administration, contraindications and/or warnings concerning the use of such therapeutic products. Suitable containers include, e.g., bottles, blister packs, etc. The container may be formed from a variety of suitable material, including plastic, for example. The container may also be a package containing a composition in the form of a tablet or capsule, the latter being preferred, where the fixed dose triethylenetetramine disuccinate is provided in a blister pack, by way of example. The label or package insert indicates that the composition is used for treating subject having (or suspecting of having) a disease, disorder or condition relating to copper excess or unwanted copper, or that is otherwise treatable with a copper chelator. In one embodiment, the instructions recite that the triethylenetetramine disuccinate is to be administered to patients with Wilson's disease previously receiving triethylenetetramine dihydrochloride or DPA. In other embodiments, the instructions recite that the triethylenetetramine disuccinate is to be administered to patients with heart failure, diabetic cardiomyopathy, left ventricular hypertrophy, cancer, or another disease, disorder or condition described herein. The instructions will refer to one or more of the doses or dosing regimens described herein.

In another embodiment of the invention, an article of manufacture containing a dose or doses of triethylenetetramine disuccinate useful for the treatment of the disorders described herein. The article of manufacture comprises a container, a label and a package insert. Suitable containers include, for example, bottles, blister packs, etc. The containers may be formed from a variety of materials such as glass or plastic. The container holds a dose(s) of a triethylenetetramine disuccinate composition which is effective for treating the condition. The label on, or associated with, the container indicates that the triethylenetetramine disuccinate dose composition is used for treating the condition of choice. In certain embodiments, the patient has Wilson's disease. In some embodiments, the patient has previously treated Wilson's disease and does not tolerate his or her previous therapy. In certain embodiments, the patient has heart failure. In certain embodiments, the patient has diabetic cardiomyopathy. In certain embodiments, the patient has left ventricular hypertrophy. In certain embodiments, the patient has cancer. The package insert may optionally contain some or all of the clinical trial results found on clinicaltrials.gov, for example, or that are later published.

Evaluation of therapy with fixed dose triethylenetetramine disuccinate may be accomplished by reference to available copper values in mammals (including human beings). Reference herein to “elevated” in relation to the presence of copper values will include humans having at least about 10 mcg free copper/dL of serum when measured. A measurement of free copper equal to total plasma copper minus ceruloplasmin-bound copper can be made using various procedures. A preferred procedure is disclosed in the Merck & Co datasheet (www.Merck.com) for SYPRINE (trientine hydrochloride) capsules, a compound used for treatment of Wilson's Disease, in which a 24-hour urinary copper analysis in is undertaken to determine free cooper in the serum by calculating the difference between quantitatively determined total copper and ceruloplasmin-copper.

### EXAMPLES

The inventions are related to and describe methods relating to discoveries surrounding the invention of the fixed doses of triethylenetetramine disuccinate described and claimed herein.

The following examples are put forth so as to provide those of ordinary skill in the art with a complete disclosure and description of how to make and use the present invention, and are not intended to limit the scope of what the inventors regard as their invention, nor are they intended to represent that the experiments below are all or the only experiments.

### Example 1 Describes

Example 1 describes an in vitro study using standard assays which predicted that triethylenetetramine disuccinate will have good absorption in humans (estimated at approximately 70%).

Example 2 is a quantitative in vivo study on the tissue distribution of triethylenetetramine disuccinate following oral administration to male albino and male pigmented rats. Significant tissue penetration was found throughout 42 different body tissues, including the brain, heart, lung and liver, etc. in both species. In the male pigmented rat, maximum tissue concentrations of radioactivity were evenly distributed between the 1 h and 8 h time points. Highest levels of radioactivity were seen in the various tissues that included the lung at 1 hr post-dose, with penetration to the lung continuing for a full 8 hours. At 24 h post-dose elimination was on-going in the male pigmented rat with approximately half of the measured tissues having levels of radioactivity below the limit of quantification. At 72 h post-dose, elimination of radioactivity in the male pigmented rat was almost complete with approximately 65% of tissues below the limit of quantification.

Novel dosing regimens for the copper-depriving compound triethylenetetramine disuccinate are described in Examples 3 and 4, which describe human population pharmacokinetic and pharmacodynamic modeling of triethylenetetramine, its two major metabolites, and copper excretion after oral 2-way crossover administration of triethylenetetramine disuccinate and triethylenetetramine dihydrochloride to healthy adult volunteers, and reveals the bioavailability of triethylenetetramine disuccinate.

### Example 1

### Evaluation of Pgp Involvement in Compound Permeability Through the Use of Caco-2 In Vitro Model for Oral Bioavailability

The presence of p-glycoprotein (Pgp) efflux pumps in mammalian intestine tissue have been previously demonstrated and play a key role in the active transport mechanisms of drugs.

The aim of this study was to evaluate the implication of Pgp in the permeability and metabolism of a test compound, the triethylenetetramine disuccinate (PX811019), using the Caco-2 in vitro model for the human intestinal barrier.

As a highly soluble and low toxicity compound in vitro, and based upon the in vitro Papp value described below, it is predicted that triethylenetetramine disuccinate will have good absorption in humans (estimated at approximately 70%).

The most commonly used models in intestinal transport studies are human intestinal cell lines, specifically the HT29 and Caco-2 cell lines, derived from colon carcinoma (Wils P., et al. Differentiated intestinal epithelial cell lines as in vitro models for predicting the intestinal absorption of drugs. Cell Biol. Toxicol. 10:393, 1994; Boulenc X. Intestinal Cell Models: Their use in evaluating the metabolism and absorption of xenobiotics. STP.Pharma. Sciences 7:259, 1997), and the most widely used in pharmaceutical research to evaluate intestinal absorption are the Caco-2 cells. Meunier V, et al. The human intestinal epithelial cell line Caco-2; Pharmacological and pharmacokinetic applications. Cell Biol. Toxicol. 11:187, 1995. Cultured on a solid permeable membrane for 21 days under conditions that enhance their polarization, this colon carcinoma-derived cell line achieves a confluent monolayer of fully differentiated cells, with apical microvilli mimicking the intestinal lumen and a completely differentiated basolateral surface, equivalent to the cell surface normally in contact with the blood system. Furthermore, they present phenotypic and physiological characteristics that closely resemble enterocytes from the human small intestine epithelium. Zweibaum A., et al. Use of cultured cell lines in studies of intestinal cell differentiation and function. In: M. Field and R. A. Frizzel (eds), Handbook of physiology. The gastrointestinal system. Vol IV: Intestinal absorption and secretion. American Physiological Society, Washington DC. 223, 1991. See Artursson P., and Karlsson J. Correlation between oral drug absorption in humans and apparent drug permeability coefficients in human intestinal epithelial (Caco-2) cells. Biochem. Biophy. Res. Com. 175:880, 1991.

The principal objective of this project was to address the Pgp involvement in metabolism and permeability of a [¹⁴C]-radiolabeled test substance, triethylenetetramine disuccinate ([2-¹⁴C]PX811019), and to further determine if unlabeled triethylenetetramine disuccinate test substance (PX811019) represents a Pgp inhibitor or substrate. To this aim, polarized cultures of Caco-2 cells which have been assessed for monolayer integrity and functionality were used as an in vitro model for the GI barrier.

Evaluation of unlabeled triethylenetetramine disuccinate (PX811019) cytotoxicity on Caco-2 cultures: To this aim, a WST-1 assay was performed. This standard assay for measuring cell proliferation, cell viability and cytotoxicity in mammalian cells utilizes measurement of mitochondrial succinate dehydrogenase activity as an index of mitochondrial damage, and is accepted as one of the most sensitive to detect early cytotoxicity events. Caco-2 cells were seeded in 96-well plates, at a density of 5×10⁵ cells/cm², such as in permeability assays. After 48 hours of culture, unlabeled PX811019 was applied at 8 different concentrations (1, 0.5, 0.25, 0.125, 0.0625, 0.0312, 0.0156, 0.0078 mM) in HBSS(x1)-Ca²⁺Mg²⁺-pH=7.4 buffer, and was incubated for 2 hours at 37° C. Cells were then checked for viability through WST-1 application and measurement of absorbance at 450 nm in an ELISA plate reader. Each concentration was tested in triplicate.

Evaluation of [2-¹⁴C] triethylenetetramine disuccinate (PX811019) permeability in Caco-2 barrier model: Once cytotoxicity to Caco-2 cultures had been assessed, [2-¹⁴C]PX811019 was incubated on 21-day Caco-2 polarized cultures in transwell filters (6.5 mm diameter; 0.4 μm pore). TEER measurement and permeability of Lucifer yellow (low permeability marker) and Antipyrin (high permeability marker) were first performed to check for barrier integrity and quality. Digoxin was used as marker to check for Pgp activity in the cultures. In parallel, the effect of test compound on barrier integrity was determined by applying unlabelled PX811019, at the concentration used in the permeability assay, together with Lucifer yellow in apical compartments of control transwell filters. For permeability assessment, [2-¹⁴C]PX811019 was then applied in donor compartments at one nontoxic concentration (1 μCi/ml; 0.02 mM) in HBSS(x1)-Ca²⁺Mg²⁺-pH=7.4 buffer, and incubated for 1 hour at 37° C., alone or in the presence of verapamil or sodium azide. Samples were recovered from receptor compartments after 0, 15, 30, 45, 60, and 120 minutes, and further analyzed by liquid scintillation counting. Samples were also recovered a time 0 and 120 minutes from the donor compartments for mass balance evaluation. Each condition was performed in 3 replicated transwell filters in the presence of the Caco-2 barrier. Based on dpm primary data obtained from sample analysis by scintillation counting, permeability coefficient (Papp in cm/s) was calculated in both A-B (apical-basal) and B-A (basal-apical) directions, and [2-¹⁴C]PX811019 permeability was evaluated under each experimental condition.

Evaluation of unlabeled triethylenetetramine disuccinate (PX811019) effect on Pgp activity in Caco-2 barrier model: Once evaluated for its permeability on Caco-2 cultures, [³H]-digoxin was incubated alone or together with unlabeled triethylenetetramine disuccinate (PX811019) on 21-day Caco-2 polarized cultures in transwell filters (6.5 mm diameter; 0.4 μm pore). TEER measurement and permeability of Lucifer yellow (low permeability marker) and Antipyrin (high permeability marker) were first performed to check for barrier integrity and quality. In parallel, the effect of test compound on barrier integrity was determined by applying unlabeled PX811019, at the concentration used in the permeability assay, together with Lucifer yellow into apical compartments of control transwell filters. To assess the effect of test compound on Pgp activity, [³H] digoxin (4 μCi/ml; 0.2 mM) was then applied alone or in the presence of a similar and nontoxic concentration of PX811019 test compound (0.2 mM) into donor compartments in HBSS(x1)-Ca²⁺Mg²⁺-pH=7.4 buffer, and incubated for 1 hour at 37° C. Samples were recovered from receptor compartments after 0, 15, 30, 45, 60, and 120 minutes, and further analyzed by liquid scintillation counting. Samples were also recovered at time 0 and 120 minutes from the donor compartments for mass balance evaluation. Each condition was performed in 3 replicated transwell filters with in the presence of the Caco-2 barrier. Based on dpm primary data obtained from sample analysis by scintillation counting, the permeability coefficient of [³H]digoxin (Papp in cm/s) was calculated in both A-B (apical-basal) and B-A (basal-apical) directions, and the effect of PX811019 on Pgp-dependent digoxin permeability was evaluated under each experimental condition.

### Main Results

Unlabeled triethylenetetramine_disuccinate (PX811019) did not present any cytotoxicity on Caco-2 cells at any of the concentrations tested.
Caco-2 polarized monolayers used in this study fulfilled the quality criteria for barrier status required for predictive in vitro permeability assay: TEER values were higher than 1000 ohm·cm²; Papp values for Lucifer yellow (low permeability marker) were lower than 1×10⁻⁶ cm/s and Papp values for Antipyrin (high permeability marker) were higher than 1×10⁻⁶ cm/s in both experiments.
Digoxin presented a low Papp value in the A-B direction (0.75±×10⁻⁶ cm/s) and medium Papp value in the B-A direction (6.08×10⁻⁶ cm/s), with an Asymmetry Index of 8.06, indicative of Pgp activity in the system. In contrast, [2-¹⁴C] triethylenetetramine disuccinate (PX811019) applied on these Caco-2 monolayers presented medium-high A-B permeability values at the nontoxic concentration tested, with a mean value of 9.87×10⁻⁶ cm/s, and no permeability was observed either in the B-A direction, or in presence of verapamil or sodium azide. Mass balance was between 70 and 120% for all the conditions tested.
In the presence of the test compound, triethylenetetramine disuccinate (PX811019), Papp values of Digoxin in either A-B or B-A directions were similar to those obtained with Digoxin alone when test compound was applied apically (0.66±0.89×10⁻⁶ cm/s and 12.7±6.76×10⁻⁶ cm/s, respectively), and both were slightly reduced when applied basolaterally (0.2±0.35×10⁻⁶ cm/s and 5.68±3.03×10⁻⁶ cm/s, respectively). However, in both situations, the Asymmetry Index was maintained and even increased. This indicates that the Digoxin transport pathway, and thus Pgp activity, were not affected by the application of the unlabeled test substance triethylenetetramine disuccinate (PX811019), independently of the compartment where it was applied.

In conclusion, triethylenetetramine disuccinate (PX811019) did not exhibit any cytotoxicity on Caco-2 cells at any of the concentrations tested (1, 0.5, 0.25, 0.125, 0.0625, 0.0312, 0.0156, 0.0078 mM). Caco-2 polarized monolayers used in this study fulfilled the quality criteria for barrier status required for predictive in vitro permeability assay: TEER values were higher than 1000 ohm·cm²; Papp Lucifer yellow was lower than 1×10⁻⁶ cm/s and Papp Antipyrin higher than 10×10⁶ cm/s. Furthermore, Papp values and Asymmetry Index obtained for Digoxin indicated that levels of Pgp activity were within an acceptable range for this cell model.

Additionally, triethylenetetramine disuccinate (PX811019) did not affect the integrity of the monolayer at the concentrations used in both assays. Trientine disuccinate (PX811019) applied on these Caco-2 monolayers presented medium-high permeability values at the concentration tested, with a mean value of 9.87×10⁻⁶ cm/s, in the absorptive (A-B) direction. As a highly soluble and low toxicity compound in vitro, and based upon the in vitro Papp value, one can predict that this compound will have good absorption in humans (estimated at approximately 70%). No permeability was observed in the secretory (B-A) direction.

Comparing with permeability data from Digoxin Pgp substrate, Papp data obtained in both A-B and B-A directions suggested that triethylenetetramine disuccinate (PX811019) crosses the Caco-2 barrier using a Pgp-independent polarized transport pathway in the absorptive direction. Triethylenetetramine disuccinate (PX811019) A-B transport through Caco-2 monolayer was fully inhibited in the presence of sodium azide and verapamil. As sodium azide is an ATP synthesis inhibitor, this suggested that test substance (PX811019) transport was ATP-dependent. Verapamil is usually used in the permeability assay as a Pgp inhibitor through inhibition of the ATP binding cassette. However, it has been extensively described as a Ca²⁺ channel blocker, more specifically L-type channels. As the A-B polarization of triethylenetetramine disuccinate transport and Asymmetry Index indicated that Pgp was not implicated, the data in the presence of verapamil would then suggest that this agent acts as a blocker of a specific transporter for triethylenetetramine disuccinate. On the basis of these data, a possible mechanism of transport through intestinal barrier of test substance could be through an active ATP-dependent and/or Ca²⁺-dependent transporter pathway.

The data obtained from the permeability assay with Digoxin alone and in presence of test substance showed that triethylenetetramine disuccinate did not affect Pgp activity. Furthermore, as it did not compete with Digoxin, it would not be a Pgp substrate, which further supports the data showing that its permeability is not Pgp-dependent in the cell model and experimental conditions used in the study.

### Example 2

### The Quantitative Tissue Distribution of Total Radioactivity in the Rat Following Single Oral Administration of [2-¹⁴C]PX811019

Study objectives: The objective of this in vivo study was to provide quantitative information on the tissue distribution of the copper-depriving compound triethylenetetramine disuccinate following oral administration to male albino and male pigmented rats. Whole body phosphor imaging (WBPI) was carried out on whole body sections taken from three albino male rats sacrificed at 1, 3, 8 and 24 h post-dose and from one pigmented rat at 1, 8, 24, 72, 168 and 336 h post-dose. Tissue radioactivity concentrations within individual sections were quantified using a phosphor imager system. Annotated images of the selected sections at each time point were produced using a supplementary software package designed for this purpose. Terminal blood samples were taken from all animals immediately prior to sacrifice and were analyzed for radioactivity. The study was conducted in compliance with Good Laboratory Practice (GLP).

Test substance: [2-¹⁴C] PX811019 (radiolabeled triethylenetetramine disuccinate), supplied by Selcia as a solid at a radiochemical purity of 99.6%. The authenticity and radiochemical purity were determined at Aptuit prior to use in this study, using high performance liquid chromatography (HPLC).

Analytical reagents: Liquid scintillant, Gold Star™, was obtained from Meridian (Epsom, Surrey, UK) and Ultima Gold™ and Permafluor®E+ were obtained from PerkinElmer LAS (UK) Ltd. The CO₂ absorbing solution Carbo Sorb®E was also obtained from PerkinElmer LAS (UK) Ltd. Unless otherwise stated, all other analytical reagents were of at least standard analytical laboratory reagent grade and were obtained principally from VWR International Ltd (Poole, Dorset, UK) and Sigma-Aldrich Company Ltd (Poole, Dorset, UK). De-ionised water was prepared in-house.

Animals: A sufficient number of animals were obtained for use in the study:

| | | |
|---|---|---|
| Species: | Rat | Rat |
| Strain: | Sprague-Dawley | Lister Hooded |
| Sex: | Male | Male |
| Age: | 6 weeks | 6 weeks |
| Number of animals: | 12 | 6 |
| Acclimatisation: | 7 days | 7 days |
| Source: | Harlan UK Limited | Harlan UK Limited |

Animals were identified uniquely by tail marking with indelible ink and animal numbers were allocated arbitrarily. All studies are conducted in accordance with the Act Animals (Scientific Procedures) Act 1986, with UK Home Office Guidance on the implementation of the Act and with all applicable Codes of Practice for the care and housing of laboratory animals. The facility used was fully accredited by the Association for Assessment and Accreditation of Laboratory Animal Care (AAALAC). The health of the animals was assessed prior to the study, and all animals were healthy and deemed suitable for experimental use.

Study design: Each rat received a single oral administration of [2-¹⁴C] PX811019 at a target dose level of 10 mg/kg free-base. Quantitative whole-body phosphor imaging (QWBPI) was carried out on whole body sections taken from three albino male rats sacrificed at 1, 3, 8 and 24 h post-dose and from one pigmented rat at 1, 8, 24, 72, 168 and 336 h post-dose. Tissue radioactivity concentrations within individual sections were quantified and annotated, and representative images of the selected sections at each time point were produced. Terminal blood samples were taken from all animals immediately prior to sacrifice and analyzed for radioactivity.

### Results

Radiochemical purity: Prior to use, the radiochemical purity of [2-¹⁴C] PX811019 was determined to be 97.7% with a single impurity of 0.9%. The mean radioactive concentration of the formulation was determined to be 22.2 μCi/g (0.82 MBq/g) at the time of dosing and the mean specific radioactivity of formulated [2-¹⁴C] PX811019 was determined to be 22.2 μCi/mg (0.82 MBq/mg).

Doses administered: The doses administered ranged between 9.96 and 10.2 mg/kg for PX811019. The radioactive dose ranged from 8.14 to 8.32 MBq/kg.

Animal observations and environmental control: No animal observations were made during the in-life phase that could be attributed to the administration of [2-¹⁴C] PX811019. During the in-life phase, the temperature and relative humidity in the room housing the animals ranged between 20° C. to 22° C. and 67% to 90%, respectively.

Tissue distribution of radioactivity following oral administration: Mean tissue concentrations of radioactivity in male albino rats following oral administration of [2-¹⁴C] PX811019 at a target dose level of 10 mg/kg free-base are presented in Table 1.

TABLE 1
Concentrations of radioactivity in organs and tissues at
various times following single oral administration of
[2-¹⁴C] PX811019 to male albino rats at a target dose
level of 10 mg/kg free-base (results expressed as ng equiv/g)

| Tissue/organ | 1 h | 3 h | 8 h | 24 h |
|---|---|---|---|---|
| LOQ (limit of | 126 | 110 | 118 | 126 |
| Adrenal cortex | 512 | 448 | 160 | blq |
| Adrenal medulla | 587 | 446 | 326 | 151 |
| Bone | 543 | 483 | 214 | blq |
| Bone marrow | 520 | 751 | 528 | 214 |
| Brain | blq | blq | blq | blq |
| Brown fat | 665 | 586 | 591 | blq |
| Caecum contents | 211281 | 437329 | 126104 | 3208 |
| Caecum wall | 22306 | 28979 | 10713 | 321 |
| Cardiac blood | 694 | 290 | 215 | blq |
| Cardiac muscle | 382 | 344 | 324 | blq |
| Epididymis | 388 | 253 | 218 | blq |
| Eye humour | blq | blq | blq | blq |
| Eye lens | blq | blq | blq | blq |
| Fur | 613 | 295 | 253 | blq |
| Harderian gland | 289 | 320 | 535 | 212 |
| Kidney cortex | 6323 | 6617 | 3783 | 1036 |
| Kidney medulla | 6760 | 4444 | 3733 | 1054 |
| Large intestine contents | blq | blq | 473267 | 9204 |
| Large intestine wall | 822 | 482 | 4964 | 605 |
| Liver | 3034 | 2231 | 1416 | 260 |
| Lung | 801 | 507 | 392 | blq |
| Nasal mucosa | blq | 332 | blq | blq |
| Pancreas | 563 | 795 | 643 | blq |
| Pineal body | 370 | 655 | 533 | 166 |
| Pituitary gland | 866 | 470 | 562 | 234 |
| Preputial gland | 411 | 519 | 380 | 201 |
| Prostate | 5361 | 6632 | 920 | blq |
| Seminal vesicles | 319 | 564 | 472 | 186 |
| Skeletal muscle | 224 | 234 | 190 | blq |
| Small intestine contents | 268536 | 96883 | 13207 | 685 |
| Small intestine wall | 14301 | 6739 | 4897 | 1144 |
| Spinal cord | blq | blq | blq | blq |
| Spleen | 520 | 573 | 547 | 231 |
| Stomach contents | 317629 | 188232 | 2601 | blq |
| Stomach wall | 2624 | 1353 | 841 | 215 |
| Submaxillary salivary | 652 | 1018 | 917 | blq |
| Testes | 196 | 159 | 184 | blq |
| Thymus | 453 | 796 | 845 | 438 |
| Thyroid gland | 433 | 671 | 683 | 287 |
| Urine | 29573 | 104341 | 15185 | 520 |
| White fat | 206 | blq | blq | blq |
| Whole blood # | 527 | 243 | 114 | 60.6 |

blq below limit of quantification
# value obtained by sample combustion

Tissue concentrations of radioactivity in male pigmented rats following oral administration of [2-¹⁴C] PX811019 at a target dose level of 10 mg/kg free-base are presented in Table 2.

TABLE 2
Concentrations of radioactivity in organs and tissues at various times following
single oral administration of [2-¹⁴C] PX811019 to male pigmented rats
at a target dose level of 10 mg/kg free-base (results expressed as ng equiv/g)

| Tissue/organ | 1 h | 8 h (114M) | 24 h | 72 h | 168 h | 336 h (118M) |
|---|---|---|---|---|---|---|
| LOQ (limit of | 125 | 123 | 114 | 129 | 112 | 106 |
| Adrenal cortex | 238 | 342 | 222 | blq | blq | blq |
| Adrenal medulla | 292 | 440 | 178 | 147 | blq | blq |
| Bone | 152 | 253 | blq | blq | blq | blq |
| Bone marrow | 292 | 368 | 210 | blq | blq | blq |
| Brain | blq | blq | blq | blq | blq | blq |
| Brown fat | 285 | 255 | 136 | blq | blq | blq |
| Caecum contents | blq | 251695 | 5099 | 226 | blq | blq |
| Caecum wall | 452 | 11591 | 1428 | 381 | blq | blq |
| Cardiac blood | 410 | 151 | blq | blq | blq | blq |
| Cardiac muscle | 241 | 151 | blq | blq | blq | blq |
| Epididymis | 303 | 151 | blq | blq | blq | blq |
| Eye Choroid layer | 324 | 157 | 128 | 144 | blq | blq |
| Eye humour | blq | blq | blq | blq | blq | blq |
| Eye lens | blq | blq | blq | blq | blq | blq |
| Fur (non-pigmented) | 249 | 277 | blq | blq | blq | blq |
| Fur (pigmented) | 307 | 299 | blq | blq | blq | blq |
| Harderian gland | 143 | 590 | 163 | 130 | blq | blq |
| Kidney cortex | 4797 | 1293 | 565 | 277 | blq | blq |
| Kidney medulla | 2648 | 1236 | 727 | 233 | blq | blq |
| Large intestine contents | ns | 424462 | 9630 | 381 | blq | blq |
| Large intestine wall | 227 | 3563 | 1369 | 192 | blq | blq |
| Liver | 1299 | 615 | 242 | blq | blq | blq |
| Lung | 480 | 194 | blq | blq | blq | blq |
| Nasal mucosa | blq | 134 | blq | blq | blq | blq |
| Pancreas | 347 | 334 | 130 | 200 | blq | blq |
| Pineal body | 255 | blq | blq | 133 | blq | blq |
| Pituitary gland | 291 | 298 | 205 | 145 | blq | blq |
| Preputial gland | 185 | 419 | 136 | blq | blq | blq |
| Prostate | ns | 277 | blq | blq | blq | blq |
| Seminal vesicles | 295 | 193 | 127 | 162 | blq | blq |
| Skeletal muscle | blq | 173 | blq | blq | blq | blq |
| Small intestine contents | 448683 | 22920 | 1771 | 167 | blq | blq |
| Small intestine wall | 4639 | 2488 | 748 | 169 | blq | blq |
| Spinal cord | blq | blq | blq | blq | blq | blq |
| Spleen | 184 | 905 | 192 | 143 | blq | blq |
| Stomach contents | 268321 | 30411 | blq | blq | blq | blq |
| Stomach wall | 1292 | 2985 | 240 | 169 | blq | blq |
| Submaxillary salivary | 448 | 651 | 142 | blq | blq | blq |
| Testes | blq | blq | blq | blq | blq | blq |
| Thymus | 268 | 572 | 204 | 155 | blq | blq |
| Thyroid gland | 375 | 472 | 161 | 134 | blq | blq |
| Urine | 6174 | 790 | 654 | 527 | blq | blq |
| White fat | 136 | blq | blq | blq | blq | blq |
| Whole blood # | 332 | 79.9 | 61.0 | 52.0 | 31.7 | 25.0 |

blq below limit of quantification
# value obtained by sample combustion

Tissue: blood ratios in male albino and pigmented rats are presented in Table 3 and Table 4, respectively.

TABLE 3
Tissue:blood ratios at various times following single
oral administration of [2-¹⁴C] PX811019 to male
albino rats at a target dose level of 10 mg/kg free-base

| Tissue/organ | 1 h | 3 h | 8 h | 24 h * |
|---|---|---|---|---|
| Adrenal cortex | 0.74 | 1.54 | 0.74 | nc |
| Adrenal medulla | 0.85 | 1.54 | 1.52 | 2.49 |
| Bone | 0.78 | 1.67 | 1.00 | nc |
| Bone marrow | 0.75 | 2.59 | 2.46 | 3.53 |
| Brain | nc | nc | nc | nc |
| Brown fat | 0.96 | 2.02 | 2.75 | nc |
| Cardiac blood | 1.00 | 1.00 | 1.00 | nc |
| Cardiac muscle | 0.55 | 1.19 | 1.51 | nc |
| Epididymis | 0.56 | 0.87 | 1.01 | nc |
| Eye humour | nc | nc | nc | nc |
| Eye lens | nc | nc | nc | nc |
| Fur | 0.88 | 1.02 | 1.18 | nc |
| Harderian gland | 0.42 | 1.10 | 2.49 | 3.50 |
| Kidney cortex | 9.11 | 22.8 | 17.6 | 17.1 |
| Kidney medulla | 9.74 | 15.3 | 17.4 | 17.4 |
| Liver | 4.37 | 7.69 | 6.59 | 4.29 |
| Lung | 1.15 | 1.75 | 1.82 | nc |
| Nasal mucosa | nc | 1.14 | nc | nc |
| Pancreas | 0.81 | 2.74 | 2.99 | nc |
| Pineal body | 0.53 | 2.26 | 2.48 | 2.74 |
| Pituitary gland | 1.25 | 1.62 | 2.61 | 3.86 |
| Preputial gland | 0.59 | 1.79 | 1.77 | 3.32 |
| Prostate | 7.72 | 22.9 | 4.28 | nc |
| Seminal vesicles | 0.46 | 1.94 | 2.20 | 3.07 |
| Skeletal muscle | 0.32 | 0.81 | 0.88 | nc |
| Spinal cord | nc | nc | nc | nc |
| Spleen | 0.75 | 1.98 | 2.54 | 3.81 |
| Submaxillary salivary gland | 0.94 | 3.51 | 4.27 | nc |
| Testes | 0.28 | 0.55 | 0.86 | nc |
| Thymus | 0.65 | 2.74 | 3.93 | 7.23 |
| Thyroid gland | 0.62 | 2.31 | 3.18 | 4.74 |
| White fat | 0.30 | nc | nc | nc |

Tissue: blood ratios calculated using cardiac blood values
nc not calculable
* calculated using whole blood value

TABLE 4
Tissue:blood ratios at various times following single
oral administration of [2-¹⁴C] PX811019 to male
pigmented rats at a target dose level of 10 mg/kg free-base

| Tissue/organ | 1 h | 8 h | 24 h * | 72 h * | 168 h | 336 h |
|---|---|---|---|---|---|---|
| Adrenal cortex | 0.58 | 2.26 | 3.64 | nc | nc | nc |
| Adrenal medulla | 0.71 | 2.91 | 2.92 | 2.83 | nc | nc |
| Bone | 0.37 | 1.68 | nc | nc | nc | nc |
| Bone marrow | 0.71 | 2.44 | 3.44 | nc | nc | nc |
| Brain | nc | nc | nc | nc | nc | nc |
| Brown fat | 0.70 | 1.69 | 2.23 | nc | nc | nc |
| Cardiac blood | 1.00 | 1.00 | nc | nc | nc | nc |
| Cardiac muscle | 0.59 | 1.00 | nc | nc | nc | nc |
| Epididymis | 0.74 | 1.00 | nc | nc | nc | nc |
| Eye Choroid layer | 0.79 | 1.04 | 2.10 | 2.77 | nc | nc |
| Eye humour | nc | nc | nc | nc | nc | nc |
| Eye lens | nc | nc | nc | nc | nc | nc |
| Fur(non- | 0.61 | 1.83 | nc | nc | nc | nc |
| Fur (pigmented) | 0.75 | 1.98 | nc | nc | nc | nc |
| Harderian gland | 0.35 | 3.91 | 2.67 | 2.50 | nc | nc |
| Kidney cortex | 11.7 | 8.56 | 9.26 | 5.33 | nc | nc |
| Kidney medulla | 6.46 | 8.19 | 11.9 | 4.48 | nc | nc |
| Liver | 3.17 | 4.07 | 3.97 | nc | nc | nc |
| Lung | 1.17 | 1.28 | nc | nc | nc | nc |
| Nasal mucosa | nc | 0.89 | nc | nc | nc | nc |
| Pancreas | 0.85 | 2.21 | 2.13 | 3.85 | nc | nc |
| Pineal body | 0.62 | nc | nc | 2.56 | nc | nc |
| Pituitary gland | 0.71 | 1.97 | 3.36 | 2.79 | nc | nc |
| Preputial gland | 0.45 | 2.77 | 2.23 | nc | nc | nc |
| Prostate | nc | 1.83 | nc | nc | nc | nc |
| Seminal vesicles | 0.72 | 1.28 | 2.08 | 3.12 | nc | nc |
| Skeletal muscle | nc | 1.15 | nc | nc | nc | nc |
| Spinal cord | nc | nc | nc | nc | nc | nc |
| Spleen | 0.45 | 5.99 | 3.15 | 2.75 | nc | nc |
| Submaxillary | 1.09 | 4.31 | 2.33 | nc | nc | nc |
| Testes | nc | nc | nc | nc | nc | nc |
| Thymus | 0.65 | 3.79 | 3.34 | 2.98 | nc | nc |
| Thyroid gland | 0.91 | 3.13 | 2.64 | 2.58 | nc | nc |
| White fat | 0.33 | nc | nc | nc | nc | nc |

Tissue:blood ratios calculated using cardiac blood values
nc not calculable
* calculated using whole blood value

As likely following oral administration, high concentrations of radioactivity were found in the gastrointestinal tract (large intestine contents 473267 ng equiv/g at 8 h post-dose, caecum contents 437329 ng equiv/g at 3 h post-dose, stomach contents 317629 ng equiv/g at 1 h post-dose and small intestine contents 268536 ng equiv/g at 1 h post-dose). Urinary concentrations were also high with the highest level being 104341 ng equiv/g observed at 3 h post-dose. All values given above are for the male albino rats and are also generally representative of the pigmented animals.

In the male albino rat, maximum tissue concentrations of radioactivity were achieved, in which cellular uptake was measured included approximately 44% of the measured tissues, at 1 h post-dose with a further 30% at 3 h post-dose. At 1 h post-dose, it appeared that absorption was on-going in approximately half of the tissues. Highest levels of radioactivity were seen in the kidney medulla, prostate, liver, pituitary gland and lung (6760, 5361, 3034, 866 and 801 ng equiv/g, respectively), compared to a cardiac blood concentration of 694 ng equiv/g. The liver and kidney medulla had their maximum concentration at this time point. At 3 h post-dose, highest levels of radioactivity were associated with the prostate, kidney cortex, kidney medulla, liver and the submaxillary salivary gland (6632, 6617, 4444, 2231 and 1018 ng equiv/g, respectively), compared to a cardiac blood concentration of 290 ng equiv/g. Although levels of radioactivity in the prostate appeared to be high at this time point it was considered that this was a result of urinary contamination. At 8 h post-dose, with the exception of the Harderian gland (535 ng equiv/g), thymus (845 ng equiv/g) and thyroid gland (683 ng equiv/g), which had their maximum tissue concentration at this time, radioactivity concentrations in tissues were lower than their maximum values. Highest levels of radioactivity were associated with the kidney cortex, kidney medulla, liver, prostate, thymus and thyroid gland (3783, 3733, 1416, 920, 845 and 683 ng equiv/g, respectively), compared to a cardiac blood concentration of 215 ng equiv/g. At 24 h post-dose, elimination of radioactivity was on-going with approximately 80% of tissues at or below the limit of quantification. Highest levels of radioactivity were observed in the kidney medulla, kidney cortex and thymus (1054, 1036 and 438 ng equiv/g, respectively).

Tissue: blood ratios in the male albino rat, where calculable, ranged between 0.28 (testes) and 22.9 (prostate) at 1 and 3 h post-dose, respectively. The majority of tissues had a tissue: blood ratio of greater than 1 with highest ratios calculated in the prostate (22.9), kidney cortex (22.8), kidney medulla (17.4) and liver (7.69) at 3, 3, 8 and 3 h post-dose, respectively. As detailed above, it was deemed that prostate levels appeared high in relation to a urinary contamination. However, the majority of tissue: blood ratios at 1 h post-dose were less than 1, with ratios tending to increase with time. This may indicate a slower uptake and release by the tissues compared with blood.

Distribution of radioactivity in the male pigmented rat was similar to that observed in the male albino rats. In the male pigmented rat, maximum tissue concentrations of radioactivity were evenly distributed between the 1 h and 8 h time points. Maximum levels were achieved in approximately 40% of the measured tissues at 1 h post-dose, with a further 45% at 8 h post-dose. At 1 h post-dose, absorption was considered to be on-going in approximately half of the tissues. Highest levels of radioactivity were seen in the kidney cortex, kidney medulla and liver (4797, 2648 and 1299 ng equiv/g, respectively), compared to a cardiac blood concentration of 410 ng equiv/g. The liver and kidney had their maximum concentration at this time point. At 8 h post-dose, highest levels of radioactivity were associated with the kidney cortex, kidney medulla, spleen, submaxillary salivary gland, liver, Harderian gland and thymus (1293, 1236, 905, 651, 615, 590 and 572 ng equiv/g, respectively), compared to a cardiac blood concentration of 151 ng equiv/g. At 24 h post-dose elimination was on-going with approximately half of the measured tissues having levels of radioactivity below the limit of quantification. Highest levels were associated with the kidney medulla and kidney cortex (727 and 565 ng equiv/g, respectively). At 72 h post-dose, elimination of radioactivity was almost complete with approximately 65% of tissues below the limit of quantification. Highest levels of radioactivity were observed in the kidney cortex, kidney medulla and pancreas (277, 233 and 200 ng equiv/g, respectively).

At 168 h post-dose, elimination appeared to be complete with all levels of radioactivity in tissues below the limit of quantification.

Tissue: blood ratios in the male pigmented rat, where calculable, ranged between 0.33 (white fat) and 11.9 (kidney medulla) at 1 and 24 h post-dose, respectively. The majority of tissues had a tissue: blood ratio of greater than 1 with highest ratios calculated in the kidney medulla (11.9), kidney cortex (11.7), spleen (5.99) and liver (4.07) at 24, 1, 8 and 8 h post-dose, respectively. However, the majority of tissue: blood ratios at 1 h post-dose were less than 1, with ratios tending to increase with time. This may indicate a slower uptake and release by the tissues compared with blood.

Radioactivity levels in blood measured by QWBPI were compared to values obtained by sample combustion of blood samples taken immediately prior to sacrifice. Similar trends and order of magnitude were evident between values obtained by QWBPI measurement and values obtained by sample combustion. Blood levels at 1 h post-dose were 694 and 527 ng equiv/g by QWBPI quantification and by sample combustion, respectively, for the male albino rats and 410 and 332 ng equiv/g by QWBPI quantification and by sample combustion, respectively, for the male pigmented animals

In summarizing certain aspects of this study, it is noted that, following oral administration, high concentrations of radioactivity were observed in the contents of the gastrointestinal tract. In the male albino rat, absorption of radioactivity was rapid with measurable levels of radioactivity present in the majority of tissues at 1 h post-dose. Maximal levels of radioactivity were reached in approximately 44% of tissues at 1 h post-dose and a further 30% of tissues reached maximum levels at 3 h post-dose. The highest tissue concentrations of radioactivity were attained in the kidney medulla, prostate, kidney cortex and liver (6760, 6632, 6617 and 3034 ng equiv/g, respectively), at 1, 3, 3 and 1 h post-dose, respectively. Although levels of radioactivity in the prostate appeared to be high it was considered that this was a result of urinary contamination.

Concentrations and distribution of radioactivity in the male pigmented rat were similar to those seen in the male albino rat. Maximum levels of radioactivity were reached in approximately 40% of tissues at 1 h post-dose and a further 45% of tissues reached maximum levels at 8 h post-dose. Highest levels were associated with the kidney cortex, kidney medulla and liver (4797, 2648 and 1299 ng equiv/g, respectively), all at 1 h post-dose.

Conclusions: The distribution and concentration of total radioactivity in the male albino rats and the male pigmented rats were similar. Binding to the melanin of pigmented tissues was not evident.

PX811019 is rapidly absorbed and distributed with nearly half of tissues having maximum concentrations of radioactivity at 1 h post-dose, but with absorption on-going in approximately half of the tissues. Tissue: blood ratios in the majority of tissues reached values greater than 1 after the 1 h time point, which may indicate a slow uptake and release by the tissues compared with blood.

Tissues associated with biotransformation and elimination (e.g., liver and kidney) and secretory glands (e.g., pancreas, submaxillary salivary gland, thymus and thyroid gland) tended to have higher concentrations of radioactivity.

Elimination of radioactivity from tissues was generally rapid, with decreased tissue levels observed at 24 h post-dose and appeared to be complete in the majority of tissues by 72 h.

### Example 3

A Single Center, Randomized, Double-Blind, Single-Dose, 2-Way Crossover, Dose Escalation Study of the Pharmacokinetics and Pharmacodynamics of Triethylenetetramine Disuccinate (PX811019) Compared with Triethylenetetramine Dihydrochloride in Normal Healthy Volunteers

This human clinical study provides population pharmacokinetic and pharmacodynamic modeling of triethylenetetramine, its two major metabolites, and copper excretion after oral 2-way crossover administration of triethylenetetramine disuccinate and triethylenetetramine dihydrochloride to healthy adult volunteers.

The population PK analysis encompasses samples from a study (TETA doses 166, 499, 832 mg of free base in each of three cohorts) where each subject received triethylenetetramine disuccinate (PX811019) and triethylenetetramine dihydrochloride (Syprine®) in a 2-way crossover design. triethylenetetramine dihydrochloride (Syprine®) is a potent copper chelator, which was approved by the FDA in 1985 for the second line treatment of Wilson's Disease. Triethylenetetramine disuccinate (PX811019) is an alternative, superior salt form of triethylenetetramine, but its target dosing is unknown, and unknowable from the prior art.

A population pharmacokinetic/pharmacodynamic (PK/PD) model was used to describe the concentrations of triethylenetetramine (TETA), its two major metabolites (monoacetylated (MAT) and diacetylated (DAT) forms), and copper excretion in urine. The model with first-order absorption and two-compartment kinetics for TETA, catenary formations of MAT and DAT, and copper excretion directly controlled by TETA in plasma was further used to identify differences between studied TETA formulations by estimating absorption-related parameters separately for PX811019 and Syprine®. The influences of subject-specific covariates and dose on PK/PD parameters were examined based on standard chi square statistics. Population PK/PD modeling was performed using the NONMEM software.

Objectives: The objectives of this study were to compare the pharmacokinetic (PK) profiles and to determine the dosing relationship of triethylenetetramine (TETA) disuccinate (PX811019) relative to TETA dihydrochloride (Syprine®) and to characterize the pharmacodynamic (PD) profile of urinary copper excretion in response to the study drug.

Abridged PK/PD, Half-Life, Absorption Kinetics and Bioavailability Summary: In carrying out this study, it was discovered that the relative bioavailability of PX811019 compared to Syprine® equaled 74.5%. Differences between Syprine and PX811019 in lag-times (0.083 and 0.239 h) and absorption rate constants (1.74 and 1.19 h⁻¹) were observed. A covariance analysis did not identify major PK/PD differences related to dose, sex, weight, or renal function. The compound exhibited highly stable and consistent PK/PD profiles. Some dose-dependence of TETA distribution volume was found producing a somewhat longer half-life at the higher doses. Differences in absorption kinetics between forms were modest with lesser bioavailability (74.5%) of PX811019 compared to Syprine® evident. It was discovered that administration of about 134% of the dose of PX811019 would produce essentially identical plasma concentrations of TETA, MAT, and DAT and copper excretion rates as Syprine®. See also, Example 3.

Methodology: This study was a Phase 1, prospective, randomized, double-blind, dose escalation, 2-way crossover design. It was planned that up to four cohorts, with six subjects per cohort, were to be enrolled. PX811019 or Syprine® doses were to be administered to subjects within each cohort at approximately molar equivalent doses of TETA free base (approximately 166 to 167 mg free base per capsule). Cohort doing is shown in Table 5:

TABLE 5
Study Dose of TETA Dihydrochloride and TETA Disuccinate

| | TETA | TETA | | |
|---|---|---|---|---|
| | Dihydrochloride | Disuccinate | | Approximate |
| | (Syprine ®) | (PX811019) | Number of | TETA mg |
| Cohort | Dose (mg) | Dose (mg) | Capsules | free base |
| 1 | 250 | 435 | 1 | 166 |
| 2 | 750 | 1305 | 3 | 499 |
| 3 | 1250 | 2175 | 5 | 832 |

Following completion of each cohort, the Sponsor and the Investigator reviewed the plasma concentration-time profiles of TETA and its acetyl metabolites [monoacetyl TETA (MAT) and diacetyl TETA (DAT)] as well as safety and tolerability data prior to approving escalation to the next cohort. Based upon analysis of interim PK data, the decision was made to stop enrollment after completion of three cohorts.

There were three visits to the clinic: a Screening Visit that occurred within 28 days prior to the first dose, and two Treatment Visits. Subjects were screened and enrolled based on medical history, clinical laboratory results, physical examination findings, vital signs assessments and resting 12-lead ECG evaluations. Eligible subjects were admitted to the research facility by 2000 h on the evening prior to dosing for each Treatment Visit. Following an overnight fast, subjects were randomized to receive a single oral dose of PX811019 or a single oral dose of Syprine® on Day 1 and the alternate treatment on Day 8. Subjects remained confined to the research facility for 48 hours following each dose (until the morning of Day 3 or Day 10).

Safety evaluations included adverse event (AE) assessments, physical examinations, clinical laboratory tests and vital sign (blood pressure and pulse rate) assessments. Blood samples for determination of plasma TETA, MAT and DAT levels were collected on Day 1 and Day 8 at Time 0 (within 30 min prior to dosing), 5, 15, 30, 60, 90, 120 min and thereafter at 3, 4, 5, 6, 8, 10 and 12 h post-dose, and then at 16, 20, 24, 30, 36, 42 and 48 h post-dose on Days 2-3 and Days 9-10. Urinary copper excretion was measured in urine collected on Day 1 and Day 8 at the following intervals: from −2-0 h pre-dose and from 0-2, 2-4, 4-6, 6-8, 8-10, 10-12 and 12-16 h post-dose and then at 16-20, 20-24, 24-30, 30-36, 36-42 and 42-48 h post-dose on Days 2-3 and Days 9-10.

Protocol Amendments: There was one protocol amendment during the study period. Protocol Amendment 1 extended the screening window from 14 to 28 days from the Screening Visit to dosing on Day 1 in order to allow sufficient time for review of safety and PK data prior to escalation to the next cohort.

Number of Subjects: A total of 18 subjects (9 male, 9 female) were enrolled and randomized to receive single oral doses of PX811019 and Syprine®.

Criteria for Inclusion: To be eligible, subjects had to complete an appropriately administered IRB-approved informed consent prior to performance of any study-related procedures, and be healthy adult males or females between the ages 18 and 60 years, inclusive, with a body mass index (BMI) between 18 and 30 kg/m², inclusive, and have normal renal function as calculated by a creatinine clearance >90 mL/min. Females of child-bearing potential had to have a negative pregnancy test at the Screening Visit and upon each admission to the research facility, be willing to use an effective means of birth control for four weeks prior to study medication administration, and be non-lactating. Males must have been willing to use effective barrier contraception for four weeks after study medication administration. Subjects were excluded if they were smokers, had a history of drug or alcohol abuse, had participated in a clinical research study within 30 days prior to the first dose of study medication or had donated 1 pint or more of blood within 56 days, or plasma within 14 days, prior to the first dose of study medication; if they used iron, copper or other dietary supplements within two weeks prior to the first dose of study medication or during the study; required prescription or over-the-counter medication or herbal or nutritional supplements within one week prior to first dose of study medication or during the study; had a history of systemic lupus erythematosus, sideroblastic anemia, dystonia, muscular spasms or myasthenia gravis, or a history of therapeutic anti-coagulation; had a known allergy to TETA or formulation excipients; had pulmonary abnormalities evident from clinical examination; or clinical laboratory results at the Screening Visit that indicated any of the following: a clinical diagnosis of iron deficiency based on levels of plasma iron, iron-binding capacity and ferritin, copper deficiency based on low levels of plasma copper or ceruloplasmin, abnormal liver function test results or a platelet count <100×10⁶/L.

Test Product, Dose, and Mode of Administration: TETA disuccinate (PX811019) was supplied as 435 mg capsules and TETA dihydrochloride (Syprine®) was supplied as 250 mg capsules, with each capsule representing approximately equimolar doses of TETA free base. Capsules for the two formulations were similar in size and shape but were not identical in appearance. In order to preserve the integrity of the blind, subjects were administered study medication while blindfolded by a designated pharmacist or sub-investigator not otherwise involved in the conduct of the study and subjects were not allowed to directly handle the capsules. Capsules were administered at approximately 0800 h on Day 1 and Day 8, following an overnight fast, with 240 mL water.

Duration of Treatment: This study included a Screening Visit within 28 days prior to the first dose of study medication administration, and two Treatment Visits separated by 7 days, each of which required 3 consecutive overnight stays.

Criteria for Evaluation: The PK profiles of PX811019 and Syprine® were evaluated by analysis of plasma concentrations of TETA and its metabolites, MAT and DAT, following single oral doses of both formulations. Pharmacodynamic parameters were evaluated by determination of urine copper excretion following single oral doses of both formulations. Safety was evaluated by assessing the frequency of treatment-emergent adverse events (AEs), discontinuations due to AEs, physical examination findings, changes in vital signs and clinical laboratory test results.

Statistical Methods: PK parameters for plasma TETA, MAT and DAT concentration data (including Cₘₐₓ, Tₘₐₓ, AUC₀₋₂₄, AUC₀₋ₜ, AUC_{0-inf}, elimination phase t½ and effective t½) were analyzed by noncompartmental methods. The dosing relationships of the PX811019 and Syprine® were evaluated by examination of the plasma concentration time curves and Cₘₐₓ for TETA, MAT and DAT for both formulations and by calculating the ratio of the AUC₀₋ₜ values for plasma TETA based on equivalent molar doses of TETA free base. Summary statistics for pharmacokinetic parameters and average urinary Cu excretion were computed for each formulation. Geometric means were also computed for AUC₀₋₂₄, AUC₀₋ₜ, AUC_{0-inf}, and Cₘₐₓ. Summary statistics (mean, median, standard error, minimum and maximum) for plasma concentrations and urinary Cu excretion were computed for each formulation at appropriate sampling times.

Safety data, including adverse events, vital signs assessments, clinical laboratory evaluations and physical examinations are summarized by formulation and dose cohort. Adverse events were coded using the MedDRA dictionary. A by-subject adverse event data listing, including verbatim term, preferred term and system organ classification, as well as severity, relationship to treatment and action taken, is provided. Concomitant medications are listed by subject and coded using the WHO drug dictionary. Descriptive statistics (arithmetic mean, standard error, median, minimum and maximum) were calculated using SAS.

Results: A total of 18 eligible subjects (9 males and 9 females) between 20 and 48 years of age, were enrolled and randomized to receive study medication. Seventeen (94.4%) subjects completed the study and one (5.6%) subject in Cohort 3 discontinued due to an adverse event following administration of PX811019 during the first Treatment Visit.

Demographics: Enrolled subjects were representative of a healthy adult population, ranging from 20 to 48 years of age. The overall mean (SD) age of enrolled subjects was 34.3 (8.14) years and the race distribution was 4 (22.2%) White, 6 (33.3%) Black, 7 (38.9%) Latino/Hispanic and 1 (5.6%) American Indian/Alaskan Native. Mean (SD) height and weight were 169.5 (8.46) cm and 73.9 (11.41) kg, respectively, and mean (SD) BMI was 25.7 (3.27) kg/m.

### Safety Results:

Treatment-Emergent AEs: Five subjects reported treatment-emergent adverse events; 3 of 17 (17.6%) subjects reported an AE after receiving Syprine® and 2 of 18 (11.1%) subjects reported an AE after receiving PX811019. AEs reported following administration of Syprine® included headache, diarrhea and nausea. AEs reported following administration of PX811019 included headache, diarrhea and elevated liver enzymes. All AEs were mild or moderate in intensity, and resolved prior to discharge from the study, and no serious AEs were reported. One subject in Cohort 3 discontinued the study due to mild, reversible elevated liver enzymes following administration of PX811019 (2175 mg) during the first Treatment Visit.

Other Safety Assessments: No clinically significant hemodynamic effects attributable to study medication were observed based on sitting blood pressure and pulse rate.

No clinically significant changes in laboratory test parameters were observed, except for one subject receiving PX811019 (2175 mg) who was reported to have a mild, reversible elevation in liver enzymes, considered possibly related to study medication.

### Pharmacokinetic Results:

The mean pharmacokinetic parameters for TETA, MAT and DAT following single equimolar oral doses in Cohorts 1, 2 and 3 are listed below in Table 6, and are further described in Example 3:

TABLE 6
Summary of PK Parameters for TETA, MAT and DAT as obtained from non-compartmental analysis.
The half-lives derived from the final PK/PD model estimates are shown for comparison.
Summary of PK Parameters for TETA, MAT and DAT in PK Population

| | Cohort 1 (N −= 6) | | Cohort 2 (N −= 6) | | Cohort 3 (N −= 6) | |
|---|---|---|---|---|---|---|
| | PX811014 | Syprine ® | PX811019 | Syprine ® | PX811019 | Syprine ® |
| Parameter (unit | 435 mg | 250 mg | 1305 mg | 750 mg | 2175 mg | 1250 mg |

TETA

| | | | | | | |
|---|---|---|---|---|---|---|
| Cₘₐₓ (mg/L) | 292.3412 | 496.6128 | 1121.0355 | 1873.0383 | 1814.5398 | 3430.2492 |
| AUC₀₋₂₄ (mg/L · h) | 1090.0472 | 1515.2096 | 4173.2842 | 6376.9208 | 7037.9122 | 13275.2109 |
| AUC₀₋ₜ (mg/L · h) | 1089.6795 | 1541.5092 | 4391.2987 | 6644.2427 | 7398.6177 | 13740.1848 |
| AUC_{0-inf} (mg/L · h) | 1157.8173 | 1691.0937 | 4738.4486 | 6905.3857 | 7728.7437 | 14131.1832 |
| t½ (h) | 8.3944 | 18.7837 | 26.8652 | 22.9857 | 21.7903 | 23.9489 |
| t½ (alpha) Predicted, h | 1.16 | 1.16 | 1.14 | 1.14 | 1.44 | 1.44 |
| t½ (beta) Predicted, h | 18.2 | 18.2 | 28.0 | 28.1 | 36.6 | 36.7 |

MAT

| | | | | | | |
|---|---|---|---|---|---|---|
| Cₘₐₓ (mg/L) | 460.0520 | 507.4967 | 1042.0688 | 1370.9102 | 1373.4790 | 1555.2304 |
| AUC₀₋₂₄ (mg/L · h) | 3606.0350 | 4548.8755 | 7771.6814 | 10240.2013 | 11764.3074 | 14279.3749 |
| AUC₀₋ₜ (mg/L · h) | 3933.3491 | 4981.8145 | 8644.9491 | 11301.4154 | 13371.4157 | 16339.2152 |
| AUC_{0-inf} (mg/L · h) | 4134.0485 | 5337.3440 | 9239.8333 | 11981.3464 | 14373.6664 | 17493.6323 |
| t½ (h) | 15.8239 | 22.3079 | 17.8289 | 18.3246 | 17.0265 | 17.7415 |
| t½ Predicted, h | 3.52 | 3.52 | 3.82 | 3.82 | 4.60 | 4.60 |

DAT

| | | | | | | |
|---|---|---|---|---|---|---|
| Cₘₐₓ (mg/L) | 95.5918 | 126.8557 | 267.0867 | 406.6258 | 397.1342 | 475.1264 |
| AUC₀₋₂₄ (mg/L · h) | 874.1022 | 1141.6082 | 2234.9524 | 3352.6220 | 3250.9811 | 4413.2536 |
| AUC₀₋ₜ (mg/L · h) | 918.6399 | 1216.1421 | 2666.4082 | 3787.3474 | 4113.1406 | 5142.3914 |
| AUC_{0-inf} (mg/L · h) | 986.8079 | 1292.5907 | 2778.4138 | 3940.5216 | 4354.1000 | 5423.7201 |
| t½ (h) | 7.1751 | 8.3329 | 10.5466 | 11.6251 | 11.9974 | 12.3562 |
| t½ Predicted, h | 0.687 | 0.687 | 0.390 | 0.390 | 0.426 | 0.426 |

### PK of TETA:

The Cₘₐₓ ratios of TETA after administration of PX811019 versus Syprine® to subjects in Cohorts 1, 2 and 3 were 0.58, 0.59 and 0.55, respectively, and the AUC₀₋ₜ and AUC_{0-inf} ratios after administration of PX811019 versus Syprine® were 0.66-0.68, 0.64-0.65, and 0.55 for subjects in Cohorts 1, 2 and 3, respectively. The AUC₀₋₂₄ ratios of TETA were also lower after PX811019 versus Syprine® for subjects in all three dose cohorts.

The mean elimination t½ of TETA after administration of PX811019 and Syprine® to subjects in Cohort 1 was 8.4 and 18.8 h, respectively, and ranged from 21.8 to 26.9 h following administration of PX811019 and Syprine® to subjects in Cohort 2 and 3. The effective t½ values were approximately one-third to one-fourth the elimination t½ values in the three dose cohorts and were not dependent on the formulation, with the exception of PX811019 in Cohort 1, which was approximately half as much (4.5 h versus 8.4 h). The median Tₘₐₓ values ranged between 1.25 h and 2.0 h for all three dose cohorts.

### PK of MAT:

The Cₘₐₓ ratios of MAT after administration of PX811019 versus Syprine® to subjects in Cohorts 1, 2 and 3 were 0.87, 0.75 and 0.91, respectively. The AUC₀-t and AUC_{0-inf} ratios after administration of PX811019 versus Syprine® were 0.74-0.76, 0.74-0.75 and 0.84, respectively. The mean t½ values for MAT were 16 and 22 h following administration of PX811019 and Syprine®, respectively, to subjects in Cohort 1, and were 17-18 h following administration of PX811019 and Syprine® to subjects in Cohorts 2 and 3. Exposure to MAT, as measured by AUC, was approximately 2-3 times higher compared to TETA at all three dose levels. Cₘₐₓ of MAT was higher than Cₘₐₓ of TETA following administration of PX811019 and Syprine® to subjects in Cohort 1, but lower for subjects Cohort 3 for both formulations. The median Tₘₐₓ for MAT was 5.0-5.5 h for both formulations for subjects in all three dose cohorts, occurring later than the Tₘₐₓ for the parent compound.

### PK of DAT:

Cₘₐₓ of DAT was generally 2- to 3-fold lower than for TETA and 3- to 4-fold lower compared to MAT. The AUCs of DAT were also lower than for both the parent drug and MAT for both formulations. The Cₘₐₓ ratio of DAT for the PX811019 formulation versus Syprine® was between 0.71 (Cohort 1) and 0.88 (Cohort 3) while the AUC ratios ranged between 0.72 (Cohort 1) and 0.84 (Cohort 3). The median Tₘₐₓ value for DAT was similar to the Tₘₐₓ for MAT (5.0 to 6.0 h).

### Pharmacodynamic Results

The majority of cupriuresis occurred during the first 6 hours following dosing for all dose groups. The level of cupriuresis increased as the dose of Syprine® increased from 250 to 1250 mg. It was approximately the same at 435 and 1305 mg PX811019 and increased at the highest dose of 2175 mg.

### Conclusions

Single oral doses of Syprine® (250, 750, 1250 mg) and PX811019 (435, 1305, 2175 mg) were both safe and well-tolerated by these healthy adult male and female subjects.

Adverse events were reported in 3 (17.6%) subjects following administration of Syprine® and in 2 (11.1%) subjects following administration of PX811019, and included headache, nausea, diarrhea and elevated liver enzymes. Adverse events were either mild or moderate in intensity, and no serious adverse events were reported.

One subject discontinued study participation due to a mild, reversible increase in liver enzymes following treatment with 2175 mg PX811019.

No clinically significant hemodynamic effects attributable to study medication were observed based on sitting blood pressure and pulse rate.

No clinically significant changes in laboratory test parameters were observed, except for one subject receiving 2175 mg PX811019 who was reported to have a mild, reversible elevation in liver enzymes, considered possibly related to study medication.

The majority of cupriuresis occurred during the first 6 h following dosing for all dose groups and cupriuresis increased as dose levels increased. No clear difference in urinary excretion of copper due to formulation was apparent.

Cₘₐₓ of TETA was 41-45% lower following a single oral dose of the PX811019 formulation at all three dose levels tested compared to administration of equimolar doses of Syprine®.

AUC₀₋ₜ and AUC_{0-inf} were 34-45% lower following a single oral dose of the PX811019 formulation at all three dose levels compared to administration of equimolar doses of Syprine®.

Values of Cₘₐₓ and AUC for the metabolites MAT and DAT were lower following a single oral dose of the PX811019 formulation at all three dose levels compared to administration of equimolar doses of Syprine®.

### Overall Conclusions

Single oral doses of PX811019 (435, 1305, 2175 mg) and Syprine® (250, 750, 1250 mg) were safe and well-tolerated by these healthy adult male and female subjects. Adverse events were either mild or moderate in intensity and no serious adverse events were reported. One subject discontinued study participation due to a mild, reversible increase in liver enzymes following treatment with 2175 mg PX811019. Cₘₐₓ of TETA was 41-45% lower and AUC₀₋ₜ and AUC_{0-inf} of TETA were 34-45% lower following a single oral dose of the PX811019 formulation at the three dose levels tested compared to administration of equimolar doses of Syprine®. There was no clear difference in the urinary excretion of copper due to the formulation.

Triethylenetetramine disuccinate 1200 mg/day, given as 600 mg twice daily, would be expected to produce a significant cupruresis effect throughout the dosing interval with minimal side effects and negligible adverse effects on serum copper levels or other laboratory test parameters.

### Example 4

### Population Pharmacokinetic and Pharmacodynamic Modeling of Triethylenetetramine

The data analyzed in this report were obtained in the Example 3 study, a double-blind, dose escalation, 2-way crossover design study comparing TETA disuccinate (PX811019) and TETA dihydrochloride (Syprine®). The Example 3 study demonstrated that administration of TETA as the disuccinate salt results in lower exposure indices (Cₘₐₓ and AUC) of TETA and its metabolites. Population-based modeling is used here to compare the absorption kinetics and provide a more global assessment of relative bioavailability of the two salt forms of TETA in the context of the enacted Example 3 study design.

The Example 3 analysis applies a model-based population analysis to the data in order to obtain an integrated assessment of the pharmacokinetics of TETA, MAT, and DAT, to further assess the pharmacodynamics of urinary excretion of copper, to consider potential covariates with the PK/PD parameters such as sex, age and dose, and in comparing the PK/PD of Syprine® and PX811019 from Example 2, particularly in regard to bioavailability.

Study Analysis: A population PK/PD model for TETA and its metabolites was developed based on data obtained from the Example 3 Study (A Single Center, Randomized, Double-Blind, Single-Dose, 2-Way Crossover, Dose Escalation Study of the Pharmacokinetics and Pharmacodynamics of Triethylenetetramine Disuccinate (PX811019) Compared with Triethylenetetramine Dihydrochloride in Normal Healthy Volunteers) in which subjects were randomized to receive either a single oral dose of PX811019 or a single oral dose of Syprine® on Day 1 and the alternate treatment on Day 8. The data from three cohorts, with six subjects per cohort, were available. For one subject in cohort 3 only Day 1 PD data were available. The PX811019 or Syprine doses were administered to subjects within each cohort at approximately molar equivalent doses of TETA free base (approximately 166 to 167 mg free base per capsule) at the doses set forth in Table 5 in Example 2.

Blood samples for determination of plasma TETA, MAT and DAT concentrations were collected on Days 1 and 8 at Time 0 (within 30 min prior to dosing), 5, 15, 30, 60, 90, 120 min and thereafter at 3, 4, 5, 6, 8, 10, 12, 16 and 20 h post-dose, and then at 24, 30, 36, 42 and 48 h post-dose on Days 2-3 and Days 9-10. Urinary copper excretion was measured via urine collections on Days 1 and 8 at the following intervals: from −2-0 h (pre-dose) and from 0-2, 2-4, 4-6, 6-8, 8-10, 10-12, 12-16, 16-20 and 20-24 h post-dose and then at 24-30, 30-36, 36-42 and 42-48 h post-dose on Days 2-3 and Days 9-10.

### Population Pharmacokinetic/Pharmacodynamic Analysis

Data Handling for PK/PD Analysis: The PK samples were analyzed using a validated bioanalytical LC/MS/MS method for the simultaneous determination of triethylenetetramine and its two main metabolites in human serum. Triethylenetetramine (TETA) and two major TETA-derived metabolites were measured: N1-acetyltriethylenetetramine (MAT) and N1,N10-diacetyltriethylenetetramine (DAT). The assay LLOQ was 0.005 mg/L for TETA, MAT and DAT. The urine samples were collected for copper analysis, which served as the pharmacodynamic endpoint. The concentrations falling below the limit of quantification (BLQ) were handled using the Beal M3 method with the F-FLAG option. Beal, SL, Ways to fit a PK model with some data below the quantification limit. J Pharmacokinet Pharmacodyn. 2001, 28:481-504.

Population PK/PD Methods: Population nonlinear mixed-effect modeling was done using NONMEM (Version 7.3.0, Icon Development Solutions, Ellicott City, MD, USA) and the gfortran compiler 9.0. NONMEM runs were executed using Wings for NONMEM (WFN730, http://wfn.sourceforge.net). The Laplacian estimation method was used. The differential equations for the model were solved using ADVAN6 PREDPP subroutines. The NONMEM data processing and plots were performed in Matlab® Software version 7.0 (The Math Works, Inc., Natick, MA, USA).

The minimum value of the NONMEM objective function, typical goodness-of-fit diagnostic plots, and the evaluation of the precision of pharmacokinetic parameter and variability estimates were used to discriminate between various models during the model-building process.

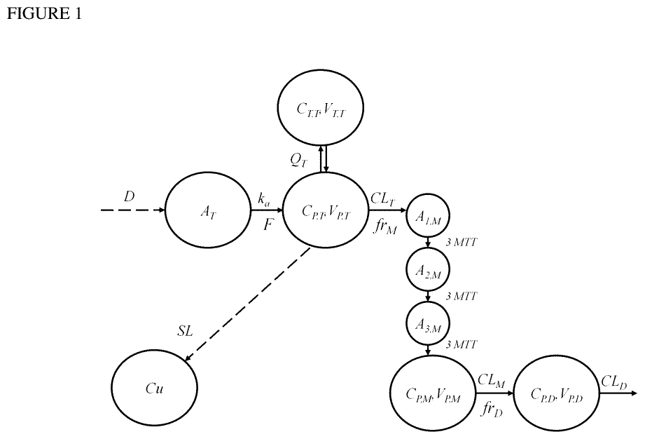

Population PK/PD Model: A PK/PD model (FIG. 1) was used to describe TETA, MAT, and DAT concentrations and copper amounts excreted in urine. It is a first-order absorption, two-compartment disposition model for TETA and catenary one-compartment disposition models for MAT and DAT. A series of transit compartments was used to describe the delay between the TETA and MAT concentrations. The following equations were used for the PK:

$$\frac{d {A}_{T}}{d t} = - {k}_{a} \cdot {A}_{T} \qquad ( Equation 1 )$$

$${V}_{P , T} \cdot \frac{d {C}_{P , T}}{d t} = CONV \cdot F \cdot {F}_{P / S} \cdot {k}_{a} \cdot {A}_{T} - {Q}_{T} \cdot {C}_{P , T} + {Q}_{T} \cdot {C}_{T , T} - C {L}_{T} \cdot {C}_{P , T} \qquad ( Equation 2 )$$

$${V}_{T , T} \cdot \frac{d {C}_{T ,}}{d t} = {Q}_{T} \cdot {C}_{P , T} - {Q}_{T} \cdot {C}_{T , T} \qquad ( Equation 3 )$$

$$\frac{d {A}_{1 , M}}{d t} = f {r}_{M} \cdot {CL}_{T} \cdot {C}_{P , T} - 3 / MTT \cdot {A}_{1 , M} \qquad ( Equation 4 )$$

$$\frac{d {A}_{2 , M}}{d t} = 3 / MTT \cdot ( {A}_{1 , M} - {A}_{2 , M} ) \qquad ( Equation 5 )$$

$$\frac{d {A}_{3 , M}}{d t} = 3 / MTT \cdot ( {A}_{2 , M} - {A}_{3 , M} ) \qquad ( Equation 6 )$$

$${V}_{P , M} \cdot \frac{{dC}_{P , M}}{d t} = 3 / MTT \cdot {A}_{3 , M} \cdot \frac{188}{146} - C {L}_{M} \cdot {C}_{P , M} \qquad ( Equation 7 )$$

$${V}_{P , D} \cdot \frac{d {C}_{P , D}}{d t} = f {r}_{D} \cdot {CL}_{M} \cdot {C}_{P , M} \frac{233}{188} - C {L}_{D} \cdot {C}_{P , D} \qquad ( Equation 8 )$$

The initial conditions of Equations 1-8 are: A_{T}(0)=D; C_{P,T}(0)=0; C_{T,T}(0)=0; C_{P,M}(0)=0; A_{1,M}(0)=0; A_{2,M}(0)=0; A_{3,M}(0)=0; and C_{P,D}(0)=0. The F denotes the presumed bioavailability of TETA (Syprine); F_{P/S} denotes relative bioavailability of PX811019 vs. Syprine; C_{P,T}, C_{P,M}, C_{P,D} are the concentrations of TETA, MAT and DAT in plasma; C_{T,T} is the concentration of TETA in the peripheral compartment; CL_{T}, CL_{M}, CL_{D} are the systemic clearances of TETA, MAT and DAT; Q_{T} is the distribution clearance of TETA; V_{P,T}, V_{P,M}, V_{P,D} are the volumes of distribution for TETA, MAT and DAT; V_{T,T} is the peripheral volume of distribution for TETA; MTT is the mean transit time accounting for the delay between TETA and MAT concentrations. The model was tested with 0 to 3 transit steps. The fr_{M} and fr_{T} are fractions of TETA metabolized to MAT and MAT metabolized to DAT. The molecular masses for TETA, MAT and DAT (146, 188 and 233 g/mol) were used to convert mass changes between parent compound and metabolites (viz. TETA equivalents). CONV equaled 0.5721 (250/435) for PX811019 and 1 for Syprine and was used to convert mass of PX811019 to Syprine equivalents.

The model actual parameters generated were: CL_{T}/F, Q_{T}/F, V_{P,T}/F, V_{T,T}/F for TETA; CL_{M}/Flf_{rM} and V_{P,M}/Flf_{rM} for MAT; and CL_{D}/Flf_{rM}/f_{rD} and V_{P,D}/Flf_{rM}/f_{rD} for DAT owing to the administration of an oral dose with uncertain bioavailability (F) and the non-identifiability of the fractions (fᵣ) reflecting conversion of TETA to MAT and MAT to TETA. The additional lag-time (t_{lag}) was used to account for the delay in the up-rising phase of TETA concentration-time profiles observed after oral dosing. Values of half-life (t_{0.5}) were calculated from these parameters.

The pharmacodynamics was modeled assuming a linear relationship between TETA plasma concentrations and urinary excretion of copper (Cho H-Y, Blum R A, Sunderland T, Cooper G J S, and Jusko W J, Pharmacokinetic and pharmacodynamics modeling of a copper-selective chelator (TETA) in healthy adults, J Clin Pharmacol 2009, 49:916-928):

$$C u ( t ) = {\int }_{{t}_{- 1}}^{t} E {R}_{0} \cdot ( 1 + SL \cdot {C}_{P , T} ( τ ) ) \cdot d τ = {\int }_{{t}_{- 1}}^{t} E {R}_{0} + E {R}_{0} SL \cdot {C}_{P , T} ( τ ) \cdot d τ \qquad ( Equation 9 )$$

where t denotes the urine collection time corresponding to each copper measurement, t₋₁ is the previous time of bladder voiding (the time range between t₋₁ and t corresponds to the urine collection interval), the dτ indicates that the variable of integration is time, ER₀ is the baseline copper excretion rate, and SL is the linear slope value relating copper excretion rate to the plasma TETA concentration.

From Equation (9) the mass of copper excreted (Cu(t)) was integrated over the rate of copper excretion for each urine collection interval. For graphical display ER(t) was approximated as an amount of copper excreted (experimental or model predicted) over the urine collection interval:

$$E R ( t ) = \frac{C u ( t )}{t - {t}_{- 1}} = \frac{{\int }_{{t}_{- 1}}^{t} E {R}_{0} \cdot ( 1 + SL \cdot {C}_{P , T} ( τ ) ) \cdot d τ}{t - {t}_{- 1}} \qquad ( Equation 10 )$$

Inter-individual variability (IIV) and inter-occasion (IOV) variability for the PK parameters were modeled assuming log normal distribution:

$${P}_{i k} = {θ}_{P} \cdot exp ( {η}_{P , i} + {κ}_{P , k} ) \qquad ( Equation 12 )$$

where Pᵢₖ is a set of PK/PD parameters for the i^{th} individual and k^{th} occasion, θ_{P} is the population estimate of PK/PD parameters, ηᵢ (ETA) is a random effect with mean 0 and variance ω², κₖ (KAPPA) is an random effect with mean 0 and variance π². A separate model for population variability for F and kₐ was assumed by estimating the inter-occasion variability for those parameters. Two levels of inter-occasion variability were assumed which corresponded to each administration of TETA. For other parameters only inter-individual variability was modeled.

In the analysis, any j^{th} observation of TETA, MAT, and DAT concentration and copper amount in urine for the i^{th} individual on the k^{th} occasion, Cᵢⱼₖ, measured at time tⱼ, was defined by:

$${C}_{P , T , i j k} = {C}_{P , T} ( {P}_{i k} , {t}_{j} ) \cdot ( 1 + {ε}_{ijk , T , prop} ) + {ε}_{ijk , T , add} \qquad ( Equation 13 )$$

$${C}_{P , M , ijk} = {C}_{P , M} ( {P}_{ik} , {t}_{j} ) \cdot ( 1 + {ε}_{ijk , M , prop} ) + {ε}_{ijk , M , add} \qquad ( Equation 14 )$$

$${C}_{P , D , i j k} = {C}_{P , D} ( {P}_{i k} , {t}_{j} ) \cdot ( 1 + {ε}_{ijk , D , prop} ) + {ε}_{ijk , D , add} \qquad ( Equation 15 )$$

$${Cu}_{ijk} = Cu ( {P}_{ik} , {t}_{j} ) \cdot ( 1 + {ε}_{ijk , Cu , prop} ) + {ε}_{ijk , Cu , add} \qquad ( Equation 16 )$$

where C_{P,T}, C_{P,M}, C_{P,D}, and Cu reflect the basic structural population model (Eq. 2, 7, 8, 9), Pᵢₖ are pharmacokinetic parameters for the i^{th} individual and k^{th} occasion (i.e. CL_{T}/F, Q/F, V_{P,T}/F, V_{T,T}/F, etc.), and ε_{ikj,add} represents the additive residual intra-individual random errors. It was assumed that εᵢⱼₖ is symmetrically distributed around means of 0, with variance denoted by σ²add and σ²prop for all PK and PD measurements. The NONMEM control stream is below:

$PROBLEM TETA
$INPUT ID TIM AMT DV CMT MDV EVID IDPERIOD IND BLQ AGEy BWkg Creat
DOSE
Form GFR M1F2 Period RACE SEQ TIME
$DATA ..\..\Data\NonmemData20161118.csv IGNORE=#
$SUBROUTINE ADVAN6 TOL=6
$MODEL
COMP = (DEPOT, DEFDOSE) ;1

| | |
|---|---|
| COMP = (PER1) | ;2 TAT |
| COMP = (PER2) | ;3 TAT |
| COMP = (MET3) | ;4 MAP |
| COMP = (MET4) | ;5 DAD |
| COMP = (CU) | ;6 DAD |
| COMP = (D1) | ;7 D1 |
| COMP = (D2) | ;8 D2 |
| COMP = (D3) | ;9 D3 |

$PK
; CONVERT PX811019 DOSE TO SYPRINE EQUIVALENTS
CONV=1
IF (FORM.EQ.0) CONV=250/437
DOSECONV=DOSE*CONV ; Syprine equivalents
FORX=1;
IF (FORM.EQ.0) FORX=THETA(1) ; relative bioavailability PX811019/SYPRINE

| | |
|---|---|
| ALAG1X = THETA(2) | ; ALAG for SYPRINE |

IF (FORM.EQ.0) ALAG1X=THETA(3) ; ALAG for PX811019

| | |
|---|---|
| KAX = THETA(4) | ; KA for SYPRINE |

IF (FORM.EQ.0) KAX=THETA(5) ; KA for PX811019
; IOV
FVAR1 = DEXP(ETA(14)) ; 1
FVAR2 = DEXP(ETA(15)) ; 2
KAVAR1 = DEXP(ETA(16)) ; 1
KAVAR2 = DEXP(ETA(17)) ; 2
IF (PERIOD.EQ.1) FVAR=FVAR1
IF (PERIOD.EQ.2) FVAR=FVAR2
IF (PERIOD.EQ.1) KAVAR=KAVAR1
IF (PERIOD.EQ.2) KAVAR=KAVAR2
; TETA
ALAG1= ALAG1X *DEXP(ETA(1))
KA = KAX *KAVAR*DEXP(ETA(2))
VT = THETA(6) *DEXP(ETA(3))
CLTM = THETA(7) *DEXP(ETA(4))
VTT=THETA(8)*(1+THETA(21)*(DOSECONV-750))*DEXP(ETA(5))
QT = THETA(9) *DEXP(ETA(6)) ; MAT
MTT = THETA(10)*DEXP(ETA(7))
VM = THETA(11)*DEXP(ETA(8))
CLMD = THETA(12)*DEXP(ETA(9))
; DAT
VD=THETA(13)*DEXP(ETA(10))
CLD=THETA(14)*DEXP(ETA(11))
; CU
BES=THETA(15)*DEXP(ETA(12))
ALP=THETA(16)*DEXP(ETA(13))
K23=QT/VT
K32=QT/VTT
K50=CLD/VD
K24=CLTM/VT
K45=CLMD/VM
K74=3/MTT
$DES
CTETA=A(2)/VT
DADT(1) = − KA*A(1)
DADT(2) = CONV*FORX*FVAR*KA*A(1) − K23*A(2) + K32*A(3) − K24*A(2)
DADT(3) = K23*A(2) − K32*A(3)
DADT(4) = K74*A(9)*188/146 − K45*A(4)
DADT(5) = K45*A(4)*230/188 − K50*A(5)
DADT(6) = BES + ALP * CTETA
DADT(7) = K24*A(2) − K74*A(7)
DADT(8) = K74*A(7) − K74*A(8)
DADT(9) = K74*A(8) − K74*A(9)
$ERROR
TETACONC=A(2)/VT
LLOQ_TETA = 5/1000
LLOQ_MAT = 5/1000 LLOQ_DAT =5/1000
IF (BLQ.EQ.0.AND.CMT.EQ.2) THEN
IPRE = A(2)/VT
IRES = DV-IPRE
W=SQRT(0.00001**2+(THETA(17)*IPRE)**2)
IWRE = (DV-IPRE)/W
Y=IPRE+W*ERR(1)
ENDIF
IF (BLQ.EQ.1.AND.CMT.EQ.2) THEN
IPRE = A(2)/VT
IRES = DV-IPRE
W=SQRT(0.00001**2+(THETA(17)*IPRE)**2)
DUM=(LLOQ_TETA-IPRE)/W
CUMD=PHI(DUM) F_FLAG=1
Y=CUMD
ENDIF
IF (BLQ.EQ.0.AND.CMT.EQ.4) THEN
IPRE = A(4)/VM
IRES = DV-IPRE
W=SQRT(0.00001**2+(THETA(18)*IPRE)**2)
IWRE = (DV-IPRE)/W
Y=IPRE+W*ERR(2)
ENDIF
IF (BLQ.EQ.1.AND.CMT.EQ.4) THEN
IPRE = A(4)/VM
IRES = DV-IPRE
W=SQRT(0.00001**2+(THETA(18)*IPRE)**2) DUM=(LLOQ_MAT-IPRE)/W
CUMD=PHI(DUM)
F_FLAG=1
Y=CUMD
ENDIF
IF (BLQ.EQ.0.AND.CMT.EQ.5) THEN
IPRE = A(5)/VD
IRES =DV-IPRE
W=SQRT(0.00001**2+(THETA(19)*IPRE)**2)
IWRE = (DV-IPRE)AV
Y=IPRE+W*ERR(3)
ENDIF
IF (BLQ.EQ.1.AND.CMT.EQ.5) THEN
IPRE = A(5)/VD
IRES =DV-IPRE
W=SQRT(0.00001**2+(THETA(19)*IPRE)**2)
DUM=(LLOQ_DAT-IPRE)/W
CUMD=PHI(DUM) F_FLAG=1
Y=CUMD
ENDIF
IF (CMT.EQ.6) THEN
IPRE = A(6)
IRES = DV-IPRE W=SQRT(0.00001**2+(THETA(20)*IPRE)**2)
IWRE = (DV-IPRE)W
Y=IPRE+W*ERR(4)
ENDIF
;TETA
$THETA (0,0.746) ; F_RELATIVE
$THETA (0,0.083) ; ALAG1_SYPRINE
$THETA (0,0.100) ; ALAG1_PX811019
$THETA (0,1.72) ; KA_SYPRINE
$THETA (0,1.20) ;KA_PX811019
$THETA (0,376.) ; VT
$THETA (0,147.) ; CLTM
$THETA (0,1160.) ; VTT
$THETA (0,42.1) ; QT
; MAT
$THETA (0,0.549) ; MTT
$THETA (0,395.) ; VM
$THETA (0,75.8) ; CLMD
;DAT
$THETA (0,179.) ; VD
$THETA (0,306.) ; CLD ; CU
$THETA (0,0.592) ; ER0
$THETA (0,22.9) ; ER0*SL
$THETA (0.001) ; VT-DOSE
; ERROR MODELS
$THETA (0,0.456) ; PROPT
$THETA (0,0.227) ; PROPM
$THETA (0,0.196) ; PROPD
$THETA (0,0.542) ; PROPCU
$OMEGA 0 FIX ; ALAG1
$OMEGA 0 FIX ; KA $OMEGA 0.0141 ; VT
$OMEGA 0.0213 ; CLTM
$OMEGA 0.0626 ; VTT $OMEGA 0 FIX ; QT
$OMEGA 0 FIX ; MTT
$OMEGA BLOCK(2) ; VM-CLMD
0.25
0.1 0.157
$OMEGA BLOCK(2) ; VD-CLD
0.694
0.1 0.127
$OMEGA 1.26 ; ER0
$OMEGA 0.282 ; ER0*SL
$OMEGA BLOCK(1) 0.20 ;FVAR
$OMEGA BLOCK(1) SAME
$OMEGA BLOCK(1) 0.60 ;KAOCC
$OMEGA BLOCK(1) SAME
$SIGMA 1. FIX ;FIX
$SIGMA 1. FIX ;FIX
$SIGMA 1. FIX ;FIX
$SIGMA 1. FIX ;FIX
$ESTIMATION METHOD=COND INTER NOABORT MAXEVAL=9999 NSIG=2
SIGL=7 PRINT=2 LAPLACIAN NUMERICAL SLOW
MSFO=
$COV UNCONDITIONAL SLOW
$TABLE ID TIME EVID IPRE IWRE IRES AMT BLQ CWRES CMT TIM MDV
NOPRINT ONEHEADER FILE=SDTAB.BLE
$TABLE ID ALAG1 KA VT VTT QT VM VD CLD BES MTT FVAR KAVAR ALP
CLTM CLMD ETA1 ETA2 ETA3 ETA4 ETA5 ETA6 ETA7 ETA8 ETA9 ETA10 ETA11
ETA12
ETA13
NOAPPEND NOPRINT ONEHEADER FILE=PATAB.BLE
$TABLE ID AGEy BWkg Creat DOSE DOSECONV GFR
NOAPPEND NOPRINT ONEHEADER FILE=COTAB.BLE
$TABLE ID M1F2 Period RACE SEQ Form
NOAPPEND NOPRINT ONEHEADER FILE=CATAB.BLE

Visual Predictive Checks: The model performance was assessed by means of Visual Predictive Checks (VPC). The VPC was calculated based on 1000 datasets simulated with the final parameter estimates [7-9]. The VPC enables the comparison of predicted versus observed data over time. In this study the 10th, 50th and 90th percentiles were used to summarize the data and VPC prediction. The VPC enables the comparison of the confidence intervals obtained from prediction with the observed data over time. When the corresponding percentile from the observed data falls outside the 90% confidence interval derived from predictions, it is an indication of a model misspecification.

Covariance Analysis: One purpose of this work was to characterize possible PK/PD differences for TETA given as PX811019 versus Syprine®. Thus, all absorption-related parameters (lag-time and kₐ) were estimated separately for each formulation. Other parameters were assumed to be identical between drug formulations, unless some contrary evidence was found during the model building process.

Other possible relationships were sought using a standard covariance analysis where individual (post-hoc) estimates of the PK/PD parameters (Eta (η) or Kappa (κ)) were plotted against available covariates (weight, age, eGFR, sex, dose, sequence, period, formulation) to identify their potential effects. If the relationship was found, all the recorded values were described by means of the following regression model:

$${P}_{i k} = {θ}_{P 1} ( 1 + {θ}_{P 2} ( {COV}_{i k} - {COV}_{m e d i a n} ) ) exp ( {η}_{P , i} + {κ}_{P , k} ) \qquad ( Equation 17 )$$

where the θ_{P1} and θ_{P2} are the regression coefficients. Continuous variables were centered around their median values, COVmedian, thus allowing θ_{P1} to represent the parameter estimate for the typical patient with median covariates. Categorical covariates (such as sex) were included in the model based on indicator variables:

$${P}_{i k} = ( \begin{matrix}{θ}_{P 1} if {IND}_{ik} = 0 \\ {θ}_{P 2} if {IND}_{ik} = 1\end{matrix} ) \cdot exp ( {η}_{P , i} + {κ}_{P , k} ) \qquad ( Equation 18 )$$

where IND is an indicator variable that has a value of 1 when the covariate is present and 0 otherwise. The difference in the minimum of the NONMEM objective function (OFV) obtained for the two hierarchical models (likelihood ratio) is approximately χ²-distributed (Mould D R, Upton R N. Basic concepts in population modeling, simulation, and model-based drug development—Part 2: Introduction to pharmacokinetic modeling methods, CPT: Pharmacometrics & Systems Pharmacology 2013, 2, e38). During the covariate search the effect of each covariate was examined by adding an appropriate equation to the base model. When the difference in OFV between the models amounted to 3.84 for one degree of freedom, it was considered to be statistically significant (at p<0.05) for the covariate to be included into the base model. This process was repeated until all significant covariates were added. Then backward elimination was performed by removing one covariate at a time. The least important covariate was dropped out from the model according to the OFV unless that difference in OFV was larger than 6.63 (corresponding to p<0.01). The final model was established when no more covariates could be excluded from the model.

### Results and Discussion

The data analyzed from the 18 subjects contained 714 plasma concentration measurements for each of TETA, MAT and DAT, and 455 copper measurements in urine. There were 124 (17.4%) TETA, 113 (15.8%) MAT, and 187 (26.2%) DAT measurements that fell below the quantification limit (BQL).

Table 7 presents a summary of the subject characteristics and the available covariates.

TABLE 7
Demographic characteristics of subjects.

| | All Subjects, | |
|---|---|---|
| Parameter, units | Median [Range] or Number | |
| Age, yr | 34 | [20-48] |
| Weight, kg | 75 | [57.3-93.6] |
| Glomerular Filtration Rate (eGFR), ml/min | 121.5 | [91.3-158.8] |

| | |
|---|---|
| Male/Female | 9/9 |

The median age of the group of 9 males and 9 females was 34 years with a range of 20 and 48 years. The body weights ranged from 57.3 to 93.6 kg. All subjects had normal kidney function with the estimated glomerular filtration rate (eGFR) within a range of 91.3 to 158.8 ml/min.

Table 6 in Example 2 provides a summary of the major exposure indices of TETA, MAT, and DAT using traditional noncompartmental (NCA) analysis. It is evident from the Cₘₐₓ and AUC values that these equimolar doses of TETA produce lower concentrations of all three compounds when administered as PX811019. However, as the NCA does not account appropriately for later time BLQ values, any parameters dependent on such (e.g. t_{0.5}) may be skewed. As this study included a range of doses and joint measurements of the parent drug, two metabolites, and copper excretion, this population-based analysis was enacted to compare the two salt forms in this global, more generalized fashion.

Initially, a PK/PD model was used to describe data from a multiple-dosing study in healthy volunteers. It is a two-compartment disposition model with first-order absorption for TETA PK. The metabolites of TETA were modeled assuming catenary metabolism (TETA→MAT→DAT). Additionally, three transit steps were used to model the delay between TETA and MAT concentrations. A one-compartment disposition model was assumed for both MAT and DAT plasma concentrations. The copper in urine was modeled as a direct linear connection to TETA plasma concentrations as found earlier. Cho, H-Y, et al., Pharmacokinetic and pharmacodynamics modeling of a copper-selective chelator (TETA) in healthy adults, J Clin Pharmacol 2009, 49:916-928.

The following modifications to the Cho, H-Y, et al., model were applied: (i) an additive part of the residual error model was not needed; (ii) estimation of inter-individual variability for ER₀ (baseline copper excretion rate) was included owing to greater variability in this study; (iii) re-parametrization of Equation (9) to ER₀ and SL·ER₀ as independent parameters; (iv) inclusion of correlations between apparent volume of distribution and clearance for MAT and DAT; (v) modelling the IOV for the absorption rate constant as part of the key purpose of this study; and (vi) dose-dependence of TETA peripheral volume of distribution was found. Each of those steps improved the model fitting considerably as judged by the NONMEM objective function and visual predictive checks.

The experimental data and model fittings for plasma concentrations of TETA, MAT, and DAT and copper excretion over the entire study were graphed for each of the subjects (Subject Graphs).

Modeling inter-occasion variability in presumed general bioavailability (F) and absorption rate constant was used as a surrogate to account for overall intra-subject variation in TETA pharmacokinetics. Such inclusion of inter-occasion variability avoids bias in the population parameter estimates. Bergstrand, M, et al., Prediction-corrected visual predictive checks for diagnosing nonlinear mixed-effects models AAPS J. 2001, 13:143-151.

The model fittings in the Subject Graphs showed that the final PK model described the measured concentrations and PD responses accurately. The typical goodness-of-fit diagnostic plots for the final model were prepared. The individual and population predictions versus observed concentrations are relatively symmetrically distributed around the line of identity, the individual weighted residuals versus individual predicted concentrations and versus time do not show any trend and are relatively uniformly distributed around zero indicating good model performance in quantifying the PK data.

The VPC plots for the TETA, MAT, and DAT concentrations and copper amounts excreted in urine were used to assess the properties of the model and fitted parameters. The VPC plots indicated that both the central tendency of the data and the variability at a particular sampling time were recaptured well as most of the data points fall within the 90% Confidence Intervals. There were no major misspecifications in the model fittings with respect to the measurements and fractions of concentrations falling below the LLOQ.

The model-fitted population PK/PD parameters for TETA and metabolites are listed in Tables 8A-8D, below:

TABLE 8
Summary of the final population PK/PD parameters (A) along with inter-subject (B), inter-
occasion (C), and residual error variance estimates (D) based on the final model.

| | | |
|---|---|---|
| Parameter, | | Estimates (% CV) |
| Units | Description | [Shrinkage] |

A. Population means

| | | | |
|---|---|---|---|
| θ-FP/S | Relative bioavailability | 0.745 | (6.0) |
| | (PX 811019/Syprine) | | |
| θ-t_{lag,} h (Syprine) | Absorption lag time for Syprine | 0.083 | (0.3) |
| θ-t_{lag,} h (PX811019) | Absorption lag time for PX811019 | 0.239 | (1.4) |
| θ-kₐ, h⁻¹ (Syprine) | Absorption rate constant for Syprine | 1.74 | (55.1) |
| θ-kₐ, h⁻¹ (PX811019) | Absorption rate constant for PX811019 | 1.19 | (31.1) |
| θ-VP, T/F, L | Apparent central volume for TETA | 326 | (15.0) |
| θ-CL_{T}/F, L/h | Apparent systemic clearance for TETA | 141 | (11.1) |
| θ-Q_{T}/F, L/h | Apparent distribution clearance for TETA | 39.2 | (12.1) |
| θ-VT, T/F, L | Apparent peripheral volume for TETA for 750 | 1210 | (11.8) |
| | mg dose (Syprine equivalents) | | |
| θ-(V_{T,T}/F-Dose), | Regression parameters in relationship between | 0.0637 | (17.3) |
| L/100 mg | the TETA dose (Syprine equivalents) and V_{T,T}/F | | |
| θ-MTT, h | Mean transit time for MAT formation | 0.381 | (34.2) |
| θ-VP, M/F/frM, L | Apparent volume for MAT | 426 | (9.8) |
| θ-CL_{M}/F/fr_{M}, L/h | Apparent systemic clearance for MAT | 76.2 | (6.5) |
| θ-VP, D/F/frM/frD, L | Apparent volume for DAT | 197 | (17.7) |
| θ-CL_{D}/F/fr_{M}/fr_{D}, L/h | Apparent systemic clearance for DAT | 306 | (9.0) |
| θ-ER₀, μg/h | Baseline copper excretion rate | 0.581 | (28.7) |
| θ-SL · ER₀, | Slope between TETA and Cu excretion rate | 24.3 | (17.4) |
| (mg/L)⁻¹ · (μg/h) | | | |

A. Inter-individual Variability

| | | |
|---|---|---|
| ω²-V_{P,T}/F, % | Inter-individual variability of V_{P,T}/F | 16.3 (27.9) [22.3] |
| ω²-CL_{T}/F, % | Inter-individual variability of CL_{T}/F | 10.3 (41.4) [22.0] |
| ω²-V_{T,T}/F, % | Inter-individual variability of V_{T,T}/F | 13.5 (33.6) [30.9] |
| ω²-VP, M/F/frM, % | Inter-individual variability of V_{P,M}/F/f_{M} | 55.1 (17.4) [2.7] |
| ω²-CL_{M}/F/fr_{M}, % | Inter-individual variability of CL_{M}/F/f_{M} | 41.5 (15.1) [2.9] |
| ω²-CL_{D}/F/fr_{M}/fr_{D}, % | Inter-individual variability of CL_{D}/F/f_{M}/f_{D} | 55.9 (18.1) [1.5] |
| ω²-VP, D/F/frM/frD, % | Inter-individual variability of V_{P,D}/F/f_{M}/f_{D} | 86.8 (29.4) [2.7] |
| ω²-ER₀, % | Inter-individual variability of ER₀ | 112 (24.6) [5.9] |
| ω²-SL · ER₀, % | Inter-individual variability of SL · ER₀ | 58.1 (25.1) [7.8] |
| Cor₁ | Correlation between V_{P,M}/F/fr_{M} and | 0.927 |
| | CL_{M}/F/fr_{M}) | |
| Cor₂ | Correlation between V_{P,D}/F/fr_{M}/fr_{D} and | 0.789 |
| | CLD/F/frM/frD) | |

B. Inter-Occasion Variability

| | | |
|---|---|---|
| π²-kₐ, % | Inter-occasion variability of kₐ | 69.4 (57.6) [16.3] |
| π²-F, % | Inter-occasion variability of F | 43.7 (17.3) [5.5] |

C. Residual variability

| | | |
|---|---|---|
| σ²prop, T, % | Proportional residual error variability for TETA | 38.1 (7.5) [2.6] |
| σ²prop, D, % | Proportional residual error variability for MAT | 23.8 (7.3) [4.9] |
| σ²prop, M, % | Proportional residual error variability for DAT | 19.0 (4.0) [4.7] |
| σ²prop, Cu, % | Proportional residual error variability for Cu | 56.6 (5.4) [1.8] |

All of the PK/PD parameters, inter-subject, inter-occasion and residual error variances were estimated well with CV values smaller than 57.6%. The apparent mean central volume of distribution was 326 L for TETA, 426 L for MAT, and 197 L for DAT. The corresponding apparent clearances were 141, 76.2, and 306 L/h. For TETA the apparent peripheral volume was 1210 L for the 750 mg dose and the apparent distribution clearance was 39.2 L/h. The relative bioavailability of PX811019 versus Syprine® was 74.5%. The absorption rate constant was 1.19 h⁻¹ for PX811019 and 1.74 h⁻¹ for Syprine® and time-lags were 0.239 and 0.083 h. The mean transit time (MTT) relating TETA conversion to MAT was 0.381 h reflecting a brief delay in appearance of MAT. The baseline copper excretion rate was 0.581 μg/h. Copper excretion increased linearly in relation to TETA concentrations with a slope (SL) of 41.8 (mg/L)⁻¹.

The inter-individual variability (IIV) was generally modest to moderate and could be identified for all of the central volumes of distribution (16.3%, 55.1%, and 86.8% for TETA, MAT and DAT), all apparent systemic clearances (10.3%, 41.5% and 55.9% for TETA, MAT and DAT), for the volume of peripheral compartment for TETA (13.5%), for ER₀ (112%) and for the SL. ER₀ (58.1%). For other parameters the IIV was fixed to zero as it either tended to zero during the model-building process or was estimated with a large (>50%) shrinkage.

The repeated administration of drug leads to the occurrence of IOV. This process, when included during the model building process, substantially improved model fittings. The IOV for the presumed F and absorption rate constant was moderate and equal to 44% and 70%.

A significant relationship (p=9.0224e-05, Δ OFV=15.331) was found between V_{T,T}/F and TETA dose expressed in Syprine equivalents. The model predicted V_{T,T}/F increased by 6.37% for every 100 mg difference from the 750 mg dose of TETA. This factor may be responsible for the modest increase in half-life with dose (Table 6 in Example 13). In the final model individual values of parameters with IIV were estimated precisely as indicated by very low shrinkage of less than 20% (except V_{T,T}/F for which shrinkage equaled 31%). Savic, R M and Karlsson, M O. The importance of shrinkage in empirical Bayes estimates for diagnostics: problems and solutions. AAPS J. 2009; 11:558-69. This suggests that the data are informative about the individual-predicted parameters making possible the search for other covariate relationships. Relationships between the factors of weight, age, eGFR, sex, and sequence were sought based on the ETA plots (deviation of the individual estimate from the population mean) using the individual estimates for ETA of TETA PK/PD parameters in relation to the sex of the subjects. Similarly, relationships between the factors of formulation and occasion were sought based on KAPPA plots (deviation of the estimate at a particular occasion (visit) from the individual mean PK parameter) of TETA PK/PD parameters in relation to formulation and occasion. The lack of any trend in these data indicates that these individual covariates do not account for the remaining unexplained inter-subject variability in the PK/PD parameters.

Individual TETA, MAT, and DAT concentrations and copper excretion rates versus time were also graphed jointly for 6 typical subjects. The drug and metabolite concentrations over the full study period as well as copper excretion rates appear similar and consistent for all subjects.

The model-fitted half-life values were added to Table 6 in Example 2 for comparison with the NCA values. The beta t_{0.5} for TETA increased with dose from 18 to 28 to 37 hours due to the increase in V_{T} with dose. The LLOQ of the assay was improved for this study allowing for more extended and reliable measurements during the washout phase. An increase in V_{T} such as this is usually explained by either nonlinear plasma protein binding or increased tissue binding with drug concentration. The listed t_{0.5} values for DAT and MAT in Table 6 (Example 2) reflect theoretical disposition rates that would be expected if these compounds were administered directly. Their actual terminal slopes are governed by “formation rate-limited disposition” from TETA and are determined from such in the process of joint fitting of the entirety of the data.

The PK/PD model applied to copper excretion showed a highly consistent relationship between TETA concentrations and copper excretion that superimpose for Syprine® and PX811019 for each subject across all doses. One subject had unusually high baseline and TETA-affected copper excretion rates.

Overall, the administration of TETA as the succinate salt produces generally linear properties and PK/PD profiles that are indistinguishable from TETA given as the dihydrochloride except for lower general exposures reflected as 74.5% relative bioavailability. The absorption kinetics of the two forms differ, but only slightly. The lower Cₘₐₓ and AUC values observed in preliminary analysis of these data (Example 2, Table 6) with PX811019 can be compensated for by administration of amounts of 134% of the present succinate formulation (1/0.745). The concentrations versus time 8798 of TETA that can expected after such triethylenetetramine disuccinate dose adjustments should be the same as triethylenetetramine dihydrochloride profiles.

The inventions described and claimed herein have many attributes and embodiments including, but not limited to, those set forth or described or referenced in this Detailed Disclosure. It is not intended to be all-inclusive and the inventions described and claimed herein are not limited to or by the features or embodiments identified in this Detailed Disclosure, which is included for purposes of illustration only and not restriction.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents. Reference to any applications, patents and publications in this specification is not, and may not be taken as, an acknowledgment or any form of suggestion that they constitute valid prior art or form part of the common general knowledge in any country in the world.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. Thus, for example, in each instance herein, and in embodiments or examples of the present invention, any of the terms “comprising”, “consisting essentially of”, and “consisting of” may be replaced with either of the other two terms in the specification. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are within the following claims. In addition, where features or aspects of the invention are described in terms of Markush groups, those skilled in the art will recognize that the invention is also thereby described in terms of any individual member or subgroup of members of the Markush group.

We claim:

1-52. (canceled)

53. A pharmaceutical formulation comprising a pharmaceutically acceptable carrier and a triethylenetetramine disuccinate present in an amount selected from the group consisting of about 350 mg, about 584 mg and about 701 mg of said triethylenetetramine disuccinate.

54. The pharmaceutical formulation of claim 53, further comprising a package insert wherein the package insert comprises instructions to administer the pharmaceutical formulation to a patient having a disease, condition or disorder treatable with a copper chelator.

55. The pharmaceutical formulation of claim 53, wherein the disease, condition or disorder treatable with a copper chelator is characterized by excess copper.

56. The pharmaceutical formulation of claim 54 wherein the disease, condition or disorder is selected from the group consisting of Wilson's Disease, heart failure, diabetic cardiomyopathy, diabetes mellitus, type 2 diabetes, Alzheimer's Disease, Parkinson's Disease, idiopathic pulmonary fibrosis, cancer, and copper toxicity.

57. The pharmaceutical formulation of claim 53, wherein the triethylenetetramine disuccinate has a purity of at least about 95%.

58. The pharmaceutical formulation of claim 53, wherein the triethylenetetramine disuccinate has a purity of at least about 99%.

59. The pharmaceutical formulation of claim 53, wherein the triethylenetetramine disuccinate is a crystalline form of triethylenetetramine disuccinate.

60. The pharmaceutical formulation of claim 53, wherein the triethylenetetramine disuccinate is a triethylenetetramine disuccinate anhydrate.

61. The pharmaceutical formulation of claim 53, wherein the dose of triethylenetetramine disuccinate is about 350 mg.

62. The pharmaceutical formulation of claim 53, wherein the dose of triethylenetetramine disuccinate is about 584 mg.

63. The pharmaceutical formulation of claim 53, wherein the dose of triethylenetetramine disuccinate is about 701 mg.

64. The pharmaceutical formulation of claim 53, wherein the pharmaceutical formulation is in the form of a capsule or a tablet suitable for oral administration to a human.

65. The pharmaceutical formulation of claim 53, wherein the pharmaceutical formulation is in the form of a capsule.

66. The pharmaceutical formulation of claim 53, wherein the pharmaceutical formulation is in the form of a tablet.

67. The pharmaceutical formulation of claim 64, wherein the capsule or tablet is packaged in a blister pack.

68. The pharmaceutical formulation of claim 64, wherein the capsule or tablet is packaged in a bottle.

69. The pharmaceutical formulation of claim 64, wherein the capsule or tablet is formulated to provide for a delayed release.

70. The pharmaceutical formulation of claim 53, wherein the capsule or tablet is a sustained release capsule or tablet.

71. The pharmaceutical formulation of claim 53, wherein the capsule or tablet comprises a compound that provides for improved absorption of the triethylenetetramine disuccinate.

72. A method of managing a subject with a disease treatable with a copper chelator, the method comprising administering to said subject a pharmaceutical formulation comprising a fixed dose of a triethylenetetramine disuccinate, wherein the fixed dose of triethylenetetramine disuccinate ranges from about 350 to about 700 milligrams.

73. The method of claim 72, wherein the fixed dose of the triethylenetetramine disuccinate is about 350 mg, 400 mg, about 500 mg, about 600 mg or about 700 mg.

74. The method of claim 72, wherein the fixed doses of a triethylenetetramine disuccinate are administered to the subject in an amount ranging from about 1050 mg per day to about 2300 mg per day, about 1400 mg per day to about 3500 mg per day, about 2300 mg per day to about 2800 mg per day, about 2400 mg per day to about 3000 mg per day, and about 2800 mg per day to about 5600 mg per day.

75. The method of claim 72, wherein the triethylenetetramine disuccinate has a purity selected from: at least about 95% pure or at least 99% pure.

76. The method of claim 72, wherein the triethylenetetramine disuccinate is a crystalline form of triethylenetetramine disuccinate.

77. The method of claim 72, wherein the triethylenetetramine disuccinate is a triethylenetetramine disuccinate anhydrate.

78. The method of claim 77, wherein the triethylenetetramine disuccinate is a triethylenetetramine disuccinate polymorph.

79. The method of claim 72, wherein the pharmaceutical formulation is in the form of a capsule for oral administration.

80. The method of claim 72, wherein the pharmaceutical formulation is in the form of a tablet for oral administration.

81. The method of claim 72, wherein the subject is a human.

82. The method of claim 72, wherein one or more symptoms or diagnostic markers of the disease are reduced.

83. The method of claim 72, wherein the administering lowers copper values content and/or reduces intracellular copper in the subject

84. The method of claim 72, wherein the administering reduces total copper.

85. The method of claim 72, wherein the administering reduces intracellular copper.

86. The method of claim 72, wherein the administering produces cupruresis.

87. The method of claim 72, wherein the disease treatable with a copper chelator comprises diabetes, wherein the administration of said pharmaceutical formulation alleviates one or more symptoms of diabetes.

88. The method of claim 87, wherein the diabetes is type 2 diabetes and the symptom alleviated by administration of said pharmaceutical formulation is excess or unwanted copper.

89. The method of claim 87, wherein the diabetes is type 2 diabetes and the symptom alleviated by administration of said pharmaceutical formulation is left ventricular hypertrophy.

90. The method of claim 72, wherein the disease treatable with a copper chelator comprises diabetic cardiomyopathy.

91. The method of claim 72, wherein the disease treatable with a copper chelator comprises heart disease, wherein one or more symptoms of heart disease is alleviated by the administering.

92. The method of claim 91, wherein the heart disease comprises heart failure.

93. The method of claim 91, wherein the subject has type 2 diabetes.

94. The method of claim 91, wherein the subject has insulin resistance.

95. The method of claim 92, wherein the subject has type 2 diabetes.

96. The method of claim 92, wherein the subject has insulin resistance.

97. The method of claim 72, wherein the disease treatable with a copper chelator comprises insulin resistance.

98. The method of claim 72, wherein the disease treatable with a copper chelator comprises Wilson's Disease.

99. The method of claim 72, wherein the administering further comprises administering one or more drugs that relieve or prevent inflammation or blood vessel leak.

100. The method of claim 99, wherein the blood vessel is a capillary.

101. The method of claim 72, wherein the administering is twice per day.

102. The method of claim 72, wherein the administering is one to four times per day.

103. A pharmaceutical formulation comprising triethylenetetramine disuccinate and an inhibitor selected from the following inhibitors: an inhibitor of N-acetylaminotransferase, an inhibitor of spermine/spermidine N-acetyltransferase (SSAT1), and an inhibitor of spermine/spermidine N-acetyltransferase (SSAT2).

104. A pharmaceutical formulation comprising triethylenetetramine disuccinate and a promoter of polyamine membrane transport including bergamottin, maringenin, quercetin, other psoralens, piperine, or tetrahydro-piperine that act as enhancers of membrane permeability for increased absorption.

105. A pharmaceutical formulation according to any one of claim 53, 103, or 104, wherein the pharmaceutical formulation comprising triethylenetetramine disuccinate is a capsule or tablet that has a shelf-life term of at least about 12 months at room temperature.

106. The pharmaceutical formulation according to claim 105, wherein the minimum purity of the triethylenetetramine disuccinate over said shelf-life term is least about 98.5% with no degradation product above about 0.5% and no new, unidentified impurities above about 0.1%.

107. The pharmaceutical formulation according to claim 105, wherein the triethylenetetramine disuccinate is a crystalline anhydrous form of the triethylenetetramine disuccinate, the shelf-life term is about 12 months, and the triethylenetetramine disuccinate drug substance remains within impurity specifications for about 12 months.

108. The pharmaceutical formulation according to claim 105, wherein the triethylenetetramine disuccinate is a crystalline anhydrous form of the triethylenetetramine disuccinate, has a shelf-life term at room temperature from about 12 months to about 5 years.

109. A plurality of single dose triethylenetetramine disuccinate capsules or tablets for daily administration, wherein the total daily dose is selected from the group consisting of from about 1050 mg per day to about 2300 mg per day, about 1400 mg per day to about 3500 mg per day, about 2300 mg per day to about 2800 mg per day, and about 2800 mg per day to about 5600 mg per day of a triethylenetetramine disuccinate.

110. The plurality of single dose capsule or tablets according to claim 109, wherein the plurality of single dose capsule or tablets are within a container.

111. The plurality of single dose capsule or tablets according to claim 109, wherein the container comprises a plastic bottle, a glass bottle, or a blister pack.

112. The plurality of single dose capsule or tablets according to claim 109, wherein the container comprises a package insert.

113. A pharmaceutical formulation comprising a fixed dose of about 350 mg, about 584 mg or about 701 mg triethylenetetramine disuccinate in a mucoadhesive formulation.

114. The pharmaceutical formulation of claim 113, wherein the mucoadhesive formulation comprises a compound selected from the group consisting of polymeric acrylic esters, methacrylic esters, hydroxylated methacrylic polymers, polyacrylic acid, alginate, polymethacrylic acid, chitosan, cyanoacrylates, hyaluronic acid, hydroxypropyl celluloses, gellan, polycarbopol, sodium carboxymethylcelluloses, mucin, gelatin, polycarbophil, and poloxamers.

115. The pharmaceutical formulation of claim 114, wherein the mucoadhesive formulation is a nasal muco-adhesive formulation.

116. The pharmaceutical formulation of claim 115, wherein nasal muco-adhesive formulation comprises a compound selected from the group consisting of copolymers of methyl vinyl ether, (hydroxypropyl)methylcellulose, sodium carboxymethylcellulose, carbopol-934P and Eudragit RL-10.

117. A pharmaceutical formulation comprising a fixed dose of about 350 mg, about 584 mg or about 701 mg triethylenetetramine disuccinate in a stomach retentive formulation.

118. A pharmaceutical formulation according to claim 117, wherein the triethylenetetramine disuccinate is substantially pure.

119. The pharmaceutical formulation according to claim 118, wherein the triethylenetetramine disuccinate is at least about 90% pure, at least about 95% pure, or about 100% pure.

120. The pharmaceutical formulation according to claim 117, wherein the triethylenetetramine disuccinate is triethylenetetramine disuccinate anhydrate.

121. The pharmaceutical formulation according to claim 117, wherein the triethylenetetramine disuccinate is crystalline form of triethylenetetramine disuccinate or triethylenetetramine disuccinate anhydrate.

122. The pharmaceutical formulation according to claim 119, wherein the triethylenetetramine disuccinate or triethylenetetramine disuccinate anhydrate is at least about 90% pure.

123. A method for treating a subject with type 2 diabetes for excess or unwanted copper, comprising administering about 350 mg, about 584 mg or about 701 mg triethylenetetramine disuccinate anhydrate one or more times per day, wherein said administration of said triethylenetetramine disuccinate anhydrate lowers copper(II) content in the subject.

124. A method according to claim 123, wherein said administration of said triethylenetetramine disuccinate anhydrate normalizes copper(II) content in the subject.

125. A method according to claim 123, wherein said administration of said triethylenetetramine disuccinate anhydrate maintains total copper in the subject within the normal human serum or plasma range.

126. A method according to claim 123, wherein said administration of said triethylenetetramine disuccinate anhydrate maintains total copper in the subject within at least about 70%, within at least about 75%, within about 75% to about 85%, or about 85% to about 95% of the normal range of copper in human plasma or serum.

127. The method of claim 123, wherein said subject has left ventricular hypertrophy and said left ventricular hypertrophy is reduced.

128. A method for manufacturing a copper(II) chelator product for administration to a subject, comprising bringing together a pharmaceutically acceptable carrier and about 350 mg, about 584 mg or about 701 mg of a triethylenetetramine disuccinate.

129. A method for treating a subject having disease, disorder or condition treatable with triethylenetetramine dihydrochloride, the method comprising administering to the subject a dose of triethylenetetramine disuccinate comprising between about 2.336 and 2.337 mg of triethylenetetramine disuccinate for every milligram of triethylenetetramine dihydrochloride in a dose used to treat said disease, disorder or condition.

130. The method according to claim 129, wherein the subject is an adult subject and the triethylenetetramine disuccinate is administered in an amount ranging from about 1,752 mg per day to about 2,920 per day.

131. The method according to claim 129, wherein the subject is an adult subject and the triethylenetetramine disuccinate is administered in an amount ranging from about 1,752 mg per day to about 4,672 per day.

132. The method according to claim 131, wherein the adult subject has Wilson's disease.

133. The method according to claim 129, wherein the subject is a pediatric subject and the triethylenetetramine disuccinate is administered in an amount ranging from about 1,168 mg per day to about 1,752 mg per day.

134. The method according to claim 129, wherein the subject is a pediatric subject and the triethylenetetramine disuccinate is administered in an amount ranging from about 1,168 mg per day to about 3,504 per day.

135. The method according to claim 134, wherein the pediatric subject has Wilson's disease.

136. The method according to claim 129, wherein the triethylenetetramine dihydrochloride is a crystalline anhydrous form of the triethylenetetramine disuccinate.
